SEQUENCE LISTING (9 sequences)

SEQ ID NO: 1 - protein, 1268 aa, Homo sapiens
  REGION 1..1268: note = human HDLBP Isoform a
MSSVAVLTQESFAEHRSGLVPQQIKVATLNSEEESDPPTYKDAFPPLPEKAACLESAQEPAGAWGNKIRPIKASVITQVFHVPLEERKYKDMNQFGEGEQAKICLEIMQRTGAHLELSLAKDQGLSIMVSGKLDAVMKARKDIVARLQTQASATVAIPKEHHRFVIGKNGEKLQDLELKTATKIQIPRPDDPSNQIKITGTKEGIEKARHEVLLISAEQDKRAVERLEVEKAFHPFIAGPYNRLVGEIMQETGTRINIPPPSVNRTEIVFTGEKEQLAQAVARIKKIYEEKKKKTTTIAVEVKKSQHKYVIGPKGNSLQEILERTGVSVEIPPSDSISETVILRGEPEKLGQALTEVYAKANSFTVSSVAAPSWLHRFIIGKKGQNLAKITQQMPKVHIEFTEGEDKITLEGPTEDVNVAQEQIEGMVKDLINRMDYVEINIDHKFHRHLIGKSGANINRIKDQYKVSVRIPPDSEKSNLIRIEGDPQGVQQAKRELLELASRMENERTKDLIIEQRFHRTIIGQKGERIREIRDKFPEVIINFPDPAQKSDIVQLRGPKNEVEKCTKYMQKMVADLVENSYSISVPIFKQFHKNIIGKGGANIKKIREESNTKIDLPAENSNSETIIITGKRANCEAARSRILSIQKDLANIAEVEVSIPAKLHNSLIGTKGRLIRSIMEECGGVHIHFPVEGSGSDTVVIRGPSSDVEKAKKQLLHLAEEKQTKSFTVDIRAKPEYHKFLIGKGGGKIRKVRDSTGARVIFPAAEDKDQDLITIIGKEDAVREAQKELEALIQNLDNVVEDSMLVDPKHHRHFVIRRGQVLREIAEEYGGVMVSFPRSGTQSDKVTLKGAKDCVEAAKKRIQEIIEDLEAQVTLECAIPQKFHRSVMGPKGSRIQQITRDFSVQIKFPDREENAVHSTEPVVQENGDEAGEGREAKDCDPGSPRRCDIIIISGRKEKCEAAKEALEALVPVTIEVEVPFDLHRYVIGQKGSGIRKMMDEFEVNIHVPAPELQSDIIAITGLAANLDRAKAGLLERVKELQAEQEDRALRSFKLSVTVDPKYHPKIIGRKGAVITQIRLEHDVNIQFPDKDDGNQPQDQITITGYEKNTEAARDAILRIVGELEQMVSEDVPLDHRVHARIIGARGKAIRKIMDEFKVDIRFPQSGAPDPNCVTVTGLPENVEEAIDHILNLEEEYLADVVDSEALQVYMKPPAHEEAKAPSRGFVVRDAPWTASSSEKAPDMSSSEEFPSFGAQVAPKTLPWGPKR

SEQ ID NO: 2 - protein, 1235 aa, Homo sapiens
  REGION 1..1235: note = human HDLBP Isoform b
MHLAERDRWLFVATVMMHFVSIKSGFPGLCVGVRSTMSSVAVLTQESFAEHRSGLVPQQIKVATLNSEEESDPPTYKDAFPPLPEKAACLESAQEPAGAWGNKIRPIKASVITQVFHVPLEERKYKDMNQFGEGEQAKICLEIMQRTGAHLELSLAKDQGLSIMVSGKLDAVMKARKDIVARLQTQASATVAIPKEHHRFVIGKNGEKLQDLELKTATKIQIPRPDDPSNQIKITGTKEGIEKARHEVLLISAEQDKRAVERLEVEKAFHPFIAGPYNRLVGEIMQETGTRINIPPPSVNRTEIVFTGEKEQLAQAVARIKKIYEEKANSFTVSSVAAPSWLHRFIIGKKGQNLAKITQQMPKVHIEFTEGEDKITLEGPTEDVNVAQEQIEGMVKDLINRMDYVEINIDHKFHRHLIGKSGANINRIKDQYKVSVRIPPDSEKSNLIRIEGDPQGVQQAKRELLELASRMENERTKDLIIEQRFHRTIIGQKGERIREIRDKFPEVIINFPDPAQKSDIVQLRGPKNEVEKCTKYMQKMVADLVENSYSISVPIFKQFHKNIIGKGGANIKKIREESNTKIDLPAENSNSETIIITGKRANCEAARSRILSIQKDLANIAEVEVSIPAKLHNSLIGTKGRLIRSIMEECGGVHIHFPVEGSGSDTVVIRGPSSDVEKAKKQLLHLAEEKQTKSFTVDIRAKPEYHKFLIGKGGGKIRKVRDSTGARVIFPAAEDKDQDLITIIGKEDAVREAQKELEALIQNLDNVVEDSMLVDPKHHRHFVIRRGQVLREIAEEYGGVMVSFPRSGTQSDKVTLKGAKDCVEAAKKRIQEIIEDLEAQVTLECAIPQKFHRSVMGPKGSRIQQITRDFSVQIKFPDREENAVHSTEPVVQENGDEAGEGREAKDCDPGSPRRCDIIIISGRKEKCEAAKEALEALVPVTIEVEVPFDLHRYVIGQKGSGIRKMMDEFEVNIHVPAPELQSDIIAITGLAANLDRAKAGLLERVKELQAEQEDRALRSFKLSVTVDPKYHPKIIGRKGAVITQIRLEHDVNIQFPDKDDGNQPQDQITITGYEKNTEAARDAILRIVGELEQMVSEDVPLDHRVHARIIGARGKAIRKIMDEFKVDIRFPQSGAPDPNCVTVTGLPENVEEAIDHILNLEEEYLADVVDSEALQVYMKPPAHEEAKAPSRGFVVRDAPWTASSSEKAPDMSSSEEFPSFGAQVAPKTLPWGPKR

SEQ ID NO: 3 - protein, 1250 aa, Homo sapiens
  REGION 1..1250: note = human HDLBP Isoform c
MSSVAVLTQESFAEHRSGLVPQQIKVATLNSEEESDPPTYKDAFPPLPEKAACLESAQEPAGAWGNKIRPIKASVITQVFHVPLEERKYKDMNQFGEGEQAKICLEIMQRTGAHLELSLAKDQGLSIMVSGKLDAVMKARKDIVARLQTQASATVAIPKEHHRFVIGKNGEKLQDLELKTATKIQIPRPDDPSNQIKITGTKEGIEKARHEVLLISAEQDKRAVERLEVEKAFHPFIAGPYNRLVGEIMQETGTRINIPPPSVNRTEIVFTGEKEQLAQAVARIKKIYEEKKKKTTTIAVEVKKSQHKYVIGPKGNSLQEILERTGVSVEIPPSDSISETVILRGEPEKLGQALTEVYAKANSFTVSSVAAPSWLHRFIIGKKGQNLAKITQQMPKVHIEFTEGEDKITLEGPTEDVNVAQEQIEGMVKDLINRMDYVEINIDHKFHRHLIGKSGANINRIKDQYKVSVRIPPDSEKSNLIRIEGDPQGVQQAKRELLELASRMENERTKDLIIEQRFHRTIIGQKGERIREIRDKFPEVIINFPDPAQKSDIVQLRGPKNEVEKCTKYMQKMVADLVENSYSISVPIFKQFHKNIIGKGGANIKKIREESNTKIDLPAENSNSETIIITGKRANCEAARSRILSIQKDLANIAEVEVSIPAKLHNSLIGTKGRLIRSIMEECGGVHIHFPVEGSGSDTVVIRGPSSDVEKAKKQLLHLAEEKQTKSFTVDIRAKPEYHKFLIGKGGGKIRKVRDSTGARVIFPAAEDKDQDLITIIGKEDAVREAQKELEALIQNLDNVVEDSMLVDPKHHRHFVIRRGQVLREIAEEYGGVMVSFPRSGTQSDKVTLKGAKDCVEAAKKRIQEIIEDLEAQVTLECAIPQKFHRSVMGPKGSRIQQITRDFSVQIKFPDREENAVHSTEPVVQENGDEAGEGREAKDCDPGSPRRCDIIIISGRKEKCEAAKEALEALVPVTIEVEVPFDLHRYVIGQKGSGIRKMMDEFEVNIHVPAPELQSDIIAITGLAANLDRAKAGLLERVKELQAEQEDRALRSFKLSVTVDPKYHPKIIGRKGAVITQIRLEHDVNIQFPDKDDGNQPQDQITITGYEKNTEAARDAILRIVGELEQMVSEDVPLDHRVHARIIGARGKAIRKIMDEFKVDIRFPQSGAPDPNCVTVTGLPENVEEAIDHILNLEEEYLADVVDSEALQVYMKPPAHEEAKAPSRGFVVRDAPWTASSSEKGRDDGMGPRA

SEQ ID NO: 4 - protein, 1301 aa, Drosophila melanogaster
MQAAAVMEETNNATTIEQQPIALINGQEQVANEQQPSSPTSVATPTSTTSGGTGNATPAFSYDDLFPALPANTSAQSQSGASGSTLARVTSSQKTHIVHVPCKERKSTESEKFGEGESKRICQQITKETGAQIEIVSGKNQSLTFLIKGKQSELLDARRKILMGFSTQASRQVTVPREHFRVILGKGGQRLREIERVTATRINIPSQSDESEFITIAGTKEGIAQAEQEIRQLSAEQYKKSSDRITVPKVYHPFIVGPYSENLNKLQEETGARINVPPQQVQKDEIVISGEKDAVAAAKAKVEAIYKDMEKKCSTVSVEVAKPKHRYVIGPKGSTIAEILQLTGVSVEMPPNDSPSETITLRGPQVALGNALTVVYQKSNSVKSVEINAAHWIHKYVIGRKGANMKQLEEDCPNVNVNCLEDKIKLEGDPENVDRAVAYLSEIIKNYEENFTFEVMTVNPSYYKHIIGKAGANVNRLKDELKVNINIEEREGQNNIRIEGPKEGVRQAQLELQEKIDKLENEKSKDVIIDRRLHRSIIGAKGEKIREVKDRYRQVTITIPTPQENTDIVKLRGPKEDVDKCHKDLLKLVKEIQESSHIIEVPIFKQFHKFVIGKGGANIKKIRDETQTKIDLPAEGDTNEVIVITGKKENVLEAKERIQKIQNELSDIVTEEVQIPPKYYNSIIGTGGKLISSIMEECGGVSIKFPNSDSKSDKVTIRGPKDDVEKAKVQLLELANERQLASFTAEVRAKQQHHKFLIGKNGASIRKIRDATGARIIFPSNEDTDKEVITIIGKEESVKKAREQLEAIIKECDEVTEGEVSVDPKHHKHFVAKRGFILHRISEECGGVMISFPRVGINSDKVTIKGAKDCIEAARQRIEEIVADLEAQTTIEVVIPQRHHRTIMGARGFKVQQVTFEFDVQIKFPDRDATEPVEGLTNGGSGENGGENEGQEGEQEVEKEAEQEPVRQCDVIRITGRIEKCEAAKQALLDLIPIEEELSVPFDLHRTIIGPRGANVRQFMSKHDVHVELPPSELKSDVIKVCGTPARVAEAREALVKMIEDYEADRADRELRSFVLQVDVDTEFHSKLIGRHGAVINKLRADHDVIISLPKRDEPNDRIISITGYQANAEAARDAILEIVGDPETLHREVIEIDKRIHPHLIGQRRRTIRKIIEDNKVNIKFSADDDNPNSIFISGKIEDVENVKELLFGMAEDYERDYLDNVAIAPPTIGAFLTGQGSGSDAAGASENGFVIKDAPWEKQKQAKNLTAPNTQSQEDFPHFAAGGAPVASTPITSVWGPKN

SEQ ID NO: 5 - protein, 1268 aa, Mus musculus
  REGION 1..1268: note = mouse Vigilin
MSSVAVLTQESFAEHRSGLVPQQIKVATLNSEEENDPPTYKDAFPPLPEKAACLESAQEPAGAWSNKIRPIKASVITQVFHVPLEERKYKDMNQFGEGEQAKICLEIMQRTGAHLELSLAKDQGLSIMVSGKLDAVMKARKDIVARLQTQASATVPIPKEHHRFVIGKNGEKLQDLELKTATKIQIPRPDDPSNQIKITGTKEGIEKARHEVLLISAEQDKRAVERLEVEKAFHPFIAGPYNRLVGEIMQETGTRINIPPPSVNRTEIVFTGEKEQLAQAVARIKKIYEEKKKKTTTIAVEVKKSQHKYVIGPKGNSLQEILERTGVSVEIPPSDSISETVILRGEPEKLGQALTEVYAKANSFTVSSVSAPSWLHRFIIGKKGQNLAKITQQMPKVHIEFTEGEDKITLEGPTEDVNVAQEQIEGMVKDLINRMDYVEINIDHKFHRHLIGKSGANINRIKDQYKVSVRIPPDSEKSNLIRIEGDPQGVQQAKRELLELASRMENERTKDLIIEQRFHRTIIGQKGERIREIRDKFPEVIINFPDPAQKSDIVQLRGPKNEVEKCTKYMQKMVADLVENSYSISVPIFKQFHKNIIGKGGANIKKIREESNTKIDLPAENSNSETIIITGKRANCEAARSRILSIQKDLANIAEVEVSIPAKLHNSLIGTKGRLIRSIMEECGGVHIHFPVEGSGSDTVVIRGPSSDVEKAKKQLLHLAEEKQTKSFTVDIRAKPEYHKFLIGKGGGKIRKVRDSTGARIIFPAAEDKDQDLITIIGKEDAVREAQKELEALIQNLENVVEDYMLVDPKHHRHFVIRRGQVLREIAEEYGGVMVSFPRSGTQSDKVTLKGAKDCVEAAKKRIQEIIEDLEAQVTVECAIPQKFHRSVMGPKGSRIQQITRDYNVQIKFPDREENPVHSVEPSIQENGDEAGEGREAKETDPGSPRRCDIIIISGRKEKCEAAKEALEALVPVTIEVEVPFDLHRYIIGQKGSGIRKMMDEFEVNIHVPAPELQSDTIAITGLAANLDRAKAGLLDRVKELQAEQEDRALRSFKLSVTVDPKYHPKIIGRKGAVITQIRLEHEVNIQFPDKDDGNQPQDQITITGYEKNTEAARDAILKIVGELEQMVSEDVPLDHRVHARIIGARGKAIRKIMDEFKVDIRFPQSGAPDPNCVTVTGLPENVEEAIDHILNLEEEYLADVVDSEALQVYMKPPAHEESRAPSKGFVVRDAPWTSNSSEKAPDMSSSEEFPSFGAQVAPKTLPWGPKR

SEQ ID NO: 6 - protein, 1268 aa, Cricetulus griseus
  REGION 1..1268: note = Chinese hamster Vigilin
MSSVAVLTQESFAEHRSGLVPQQIKVATLNSEEESDPPTYKDAFPPLPEKAACLESAQEPAGAWSNKIRPIKASVITQVFHVPLEERKYKDMNQFGEGEQAKICLEIMQRTGAHLELSLAKDQGLSIMVSGKLDAVMKARKDIVARLQTQASATVTIPKEHHRFVIGKNGEKLQDLELKTATKIQIPRPDDPSNQIKITGTKEGIEKARHEVLLISAEQDKRAVERLEVEKAFHPFIAGPYNRLVGEIMQETGTRINIPPPSVNRTEIVFTGEKEQLAQAVARIKKIYEEKKKKTTTIAVEVKKSQHKYVIGPKGNSLQEILERTGVSVEIPPSDSISETVILRGEPEKLGQALTEVYAKANSFTVSSVSAPSWLHRFIIGKKGQNLAKITQQMPKVHIEFTEGEDKITLEGPTEDVSVAQEQIEGMVKDLINRMDYVEINIDHKFHRHLIGKSGANINRIKDQYKVSVRIPPDSEKSNLIRIEGDPQGVQQAKRELLELASRMENERTKDLIIEQRFHRTIIGQKGERIREIRDKFPEVIINFPDPAQKSDIVQLRGPKNEVEKCTKYMQKMVADLVENSYSISVPIFKQFHKNIIGKGGANIKKIREESNTKIDLPAENSNSETIVITGKRANCEAARSRILSIQKDLANIAEVEVSIPAKLHNSLIGTKGRLIRSIMEECGGVHIHFPVEGSGSDTVVIRGPSSDVEKAKKQLLHLAEEKQTKSFTVDIRAKPEYHKFLIGKGGGKIRKVRDSTGARIIFPAAEDKEQDLITIVGKEDAVREAQKELEALIQNLDNVVEDYMLVDPKHHRHFVIRRGQVLREIAEEYGGVMVSFPRSGTQSDKVTLKGAKDCVEAAKKRIQEIIEDLEAQVTVECAIPQKFHRSVMGPKGSRIQQITRDYNVQIKFPDREENPVHSVEPSIQENGDEAGEGREAKETDPGSPRRCDIIIISGRREKCEAAKEALEALVPVTIEVEVPFDLHRYVIGQKGSGIRKMMDEFEVNIHVPAPELQSDIIAITGLVANLDRAKTGLLDRVKELQAEQEDRALRSFKLSVTVDPKYHPKIIGRKGAVITQIRLEHDVNIQFPDKDDGNQPQDQITITGYEKNTEAARDAILKIVGELEQMVSEDVPLDHRVHARIIGARGKAIRKIMDEFKVDIRFPQTGAPDPNCVTVTGLPENVEEAIDHILNLEEEYLADVVDSEALQVYMKPPAHEESKAPSKGFVVRDAPWTSNSSEKAPDMSSSEEFPSFGAQVAPKTLPWGPKR

SEQ ID NO: 7 - protein, 1286 aa, Chlorocebus sabaeus
  REGION 1..1286: note = green monkey Vigilin
MSSVAVLTQESFAEHRSGLVPQQIKEREDSTERMHPPVVSALSVATLNSEEESDPPTYKDAFPPLPEKAACLESAQEPAGAWGNKIRPIKASVITQVFHVPLEERKYKDMNQFGEGEQAKICLEIMQRTGAHLELSLAKDQGLSIMVSGKLDAVMKARKDIVARLQTQASATVAIPKEHHRFVIGKNGEKLQDLELKTATKIQIPRPDDPSNQIKITGTKEGIEKARHEVLLISAEQDKRAVERLEVEKAFHPFIAGPYNRLVGEIMQETGTRINIPPPSVNRTEIVFTGEKEQLAQAVARIKKIYEEKKKKTTTIAVEVKKSQHKYVIGPKGNSLQEILERTGVSVEIPPSDSISETVILRGEPEKLGQALTEVYAKANSFTVSSVAAPSWLHRFIIGKKGQNLAKITQQMPKVHIEFTEGEDKITLEGPTEDVNVAQEQIEGMVKDLINRMDYVEINIDHKFHRHLIGKSGANINRIKDQYKVSVRIPPDSEKSNLIRIEGDPQGVQQAKRELLELASRMENERTKDLIIEQRFHRTIIGQKGERIREIRDKFPEVIINFPDPAQKSDIVQLRGPKNEVEKCTKYMQKMVADLVENSYSISVPIFKQFHKNIIGKGGANIKKIREESNTKIDLPAENSNSETIIITGKRANCEAARSRILSIQKDLANIAEVEVSIPAKLHNSLIGTKGRLIRSIMEECGGVHIHFPVEGSGSDTVVIRGPSSDVEKAKKQLLHLAEEKQTKSFTVDIRAKPEYHKFLIGKGGGKIRKVRDSTGARVIFPAAEDKDQDLITIIGKEDAVREAQKELEALIQNLDNVVEDSMLVDPKHHRHFVIRRGQVLREIAEEYGGVMVSFPRSGTQSDKVTLKGAKDCVEAAKKRIQEIIEDLEAQVTLECAIPQKFHRSVMGPKGSRIQQITRDFNVQIKFPDREENPVHSVEPVVQENGDEAGEGREAKDSDPGSPRRCDIIIISGRKEKCEAAKEALEALVPVTIEVEVPFDLHRYVIGQKGSGIRKMMDEFEVNIHVPAPELQSDIIAITGLAANLDRAKAGLLERVKELQAEQEDRALRSFKLSVTVDPKYHPKIIGRKGAVITQIRLEHDVNIQFPDKDDGNQPQDQITITGYEKNTEAARDAILRIVGELEQMVSEDVPLDHRVHARIIGARGKAIRKIMDEFKVDIRFPQSGAPDPNCVTVTGLPENVEEAIDHILNLEEEYLADVVDSEVLQVYMKPPAHEEAKAPSRGFVVRDAPWTASSSEKAPDMSSSEEFPSFGAQVAPKTLPWGPKR

SEQ ID NO: 8 - DNA, 1281 bp, synthetic construct
  source 1..1281: mol_type = other DNA; note = Erythropoietin-Fc (EPO-FC)
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgcgagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagagttgagcccaaatctggtgacaaaactagtacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 9 - DNA, 1281 bp, synthetic construct
  source 1..1281: mol_type = other DNA; note = Erythropoietin-Fc, codon-optimized (EPO-FC opt 1)
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtccctgacaccaaagttaatttctatgcctggaagaggatggaggtcgggcagcaggccgtagaagtctggcagggccttgcccttctgtctgaagctgtcctgcggggccaggcccttcttgtcaactcttcccagccttgggagcctcttcagcttcatgtggataaagccgtctctggccttcgcagcctcaccactcttcttcgggctcttcgagcccagaaggaagccatctcccctcctgatgcggcctcagctgctcctctccgaacaatcactgctgacactttccgcaaactcttccgagtctactccaatttcctccggggaaagcttaagctgtacacaggggaggcctgcaggacaggggacagagttgagcccaaatctggtgacaaaactagtacatgccctccttgcccagcacctgaacttcttgggggaccttcagtcttcctcttccctcctaaacccaaggacaccctcatgatctcccggacccctgaggtcacatgcgtggtggtggacgtgagccacgaagaccctgaggtcaagttcaactggtacgtggacggcgtggaggtgcataatgccaagacaaagcctcgggaggagcagtacaactctacgtaccgtgtggtctctgtccttaccgtccttcaccaggactggcttaatggcaaggagtacaagtgcaaggtctccaacaaagcccttcctgcccctatcgagaaaaccatctccaaagccaaagggcagccccgagaaccacaggtgtacacccttcctccttcccgggatgagcttaccaagaaccaggtctctcttacctgccttgtcaaaggcttctatcccagcgacatcgccgtggagtgggagagcaatgggcagccggagaacaactacaagaccacgcctcctgtgcttgactccgacggccccttcttcctttactctaagctcaccgtggacaagagcaggtggcagcaggggaacgtcttctcatgctccgtgatgcatgaggctctgcacaaccactacacgcagaagtctctctccctttctcctggtaaataa

(19) United States
(12) Patent Application Publication
(10) Pub. No.: US 2024/0352500 A1
(43) Pub. Date: Oct. 24, 2024

(54) INCREASE OF PROTEIN EXPRESSION AND SECRETION BY ARTIFICIAL CO-EXPRESSION OF HDLBP/VIGILIN

(71) Applicants: Max-Delbrück-Centrum für Molekulare Medizin, Berlin (DE); Humboldt-Universität zu Berlin, Berlin (DE)

(72) Inventors: Markus Landthaler, Berlin (DE); Ulrike Zinnall, Lübeck (DE); Igor Minia, Berlin (DE)

(21) Appl. No.: 18/684,500

(22) PCT Filed: Aug. 18, 2022
(86) PCT No.: PCT/EP2022/073116

§ 371 Date: Feb. 16, 2024

(30) Foreign Application Priority Data
Aug. 18, 2021 (EP) 21191935.2

(51) Int. Cl. C12P 21/02; C07K 14/505; C07K 14/565; C07K 14/59; C07K 14/62; C12N 9/22; C12N 15/11; C12N 15/90

(52) U.S. Cl. CPC C12P 21/02; C07K 14/505; C07K 14/565; C07K 14/59; C07K 14/62; C12N 9/22; C12N 15/11; C12N 15/907; C07K 2319/30; C12N 2310/20

(57) ABSTRACT

The invention is related to a method for producing a secreted recombinant protein of interest in a cell, wherein the method comprises artificially co-expressing a protein different from the secreted recombinant protein of interest.

### TECHNICAL FIELD

The present invention relates to methods for producing a secreted recombinant protein of interest and cells to be used for the production of secreted proteins of interest.

### BACKGROUND

In eukaryotic cells, the final localization of functional protein products is largely determined by the site of their translation. While soluble proteins are translated in the cytosol, co-translational targeting to the endoplasmic reticulum (ER) enables newly synthesized proteins to enter the secretory pathway, resulting in their secretion or membrane integration.

The canonical secretory pathway initiates in the cytosol with the synthesis of the hydrophobic targeting signal (signal peptide or transmembrane domain). Subsequent binding of the signal recognition particle (SRP) to the nascent peptide results in ribosome elongation arrest and formation of the ribosome nascent chain complexes (RNCs). This allows the re-localization of the cytosolic SRP-RNC to the ER membrane via the SRP receptor and translocation of the nascent peptide to the ER lumen.

In recent years, a non-canonical SRP-independent pathway was discovered in yeast along with evidence for recruitment of the SRP to mRNA prior to ribosome engagement and SRP-independent ER targeting. This raises the possibility for the existence of yet unknown mechanisms for the recognition of membrane-bound mRNAs.

The potential role of regulatory elements in mRNA sequences for ribosome elongation arrest and nascent chain recognition are poorly understood. Several studies have identified elements within coding sequences (CDS) and 3′ untranslated regions (3′UTRs) that may distinguish ER-bound from cytosolic mRNAs. However, trans-acting factors that may be responsible for the recognition of such elements are yet unknown.

Recently, it was observed that a small subset of mRNAs encoding soluble proteins may also be localized and translated at the ER, indicating additional mechanisms regulating the fate of a localized mRNA. While there is evidence for subpools of ER-associated ribosomes that interact with pyruvate kinase in muscle, comprehensive differences in the composition, assembly and active translation states of cytosolic and ER-bound ribosomes have not been identified.

Furthermore, a novel variant of the ribosome-dependent nonsense mediated decay (NMD) pathway was discovered at the ER, hinting at a new layer of regulation for ER-bound mRNAs. In summary, translational fate of mRNAs encoding soluble and membrane proteins may be tightly regulated by trans-acting factors such as RNA-binding proteins, which could function beyond the canonical SRP-dependent model.

In biotechnology, as well as in biomedicine, the production and easy availability of secreted proteins such as therapeutic antibodies, hormones, enzymes, and others are of great economic interest. Means to optimize and enhance production of such protein-based compounds, especially those that can be used for medical purposes, is of great social and economic importance.

Accordingly, new strategies and methods are required for improved and increased production, expression and secretion of such protein-based compounds in cellular expression systems.

It is thus an object of the present invention to provide novel and advantageous methods for the expression and secretion of secreted recombinant proteins of interest in a cell.

It is another object of the present invention to provide an improved cellular expression system for expression of secreted recombinant proteins.

Furthermore, it is also an object of the present invention to provide the use of such an improved cellular expression system in a method of producing a secreted recombinant protein of interest.

### SUMMARY OF THE INVENTION

The aforementioned objects are solved by the aspects of the present invention as specified hereinafter.

According to the first aspect of the present invention, a method is provided for producing a secreted recombinant protein of interest in a cell, wherein the method comprises artificially co-expressing a protein different from the secreted recombinant protein of interest, wherein the amino acid sequence of the co-expressed protein has at least 90% sequence identity to an amino acid sequence according to SEQ ID NO: 1, or wherein the amino acid sequence of the co-expressed protein has at least 90% sequence identity to an amino acid sequence according to SEQ ID NO: 2.

In a preferred embodiment of the first aspect of the present invention, the amino acid sequence of the co-expressed protein has at least 95% sequence identity to an amino acid sequence according to SEQ ID NO: 1 or according to SEQ ID NO: 2, more preferably the amino acid sequence of the co-expressed protein has at least 98%, even more preferably at least 99% sequence identity to an amino acid sequence according to SEQ ID NO: 1 or according to SEQ ID NO: 2.

In another preferred embodiment of the first aspect of the invention, the co-expressed protein comprises an amino acid sequence according to SEQ ID NO: 1 or according to SEQ ID NO: 2, more preferably the co-expressed protein consists of the amino acid sequence according to SEQ ID NO: 1 or according to SEQ ID NO: 2.

In one preferred embodiment of the first aspect of the invention, the secreted recombinant protein of interest is suitable for protein-based therapies, more preferably the secreted recombinant protein is selected from the group consisting of an antibody, such as a therapeutic antibody, or an antigen-binding fragment thereof, or a nanobody, or an antibody-drug conjugate, or a recombinant fusion protein, or an exosome, or a cytokine, such as IFN-β, or a hormone, such as insulin, or a hormone analogue, such as a GnRH analogue.

In a preferred embodiment of the first aspect of the present invention, the cell is a eukaryotic cell.

In another preferred embodiment of the first aspect of the present invention, the cell is selected from the group consisting of: CHO cells, BHK 21 cells, HEK293 cells, C127, A549, Sp2/0, YB2/0, SF-9 cells, NS0 cells, Vero cells, and any derivatives thereof.

In one preferred embodiment of the first aspect of the present invention, the method is carried out in vitro.

According to the second aspect of the present invention, a eukaryotic cell is provided, wherein the cell is modified to produce a secreted recombinant protein of interest, wherein the cell artificially co-expresses a protein different from the secreted recombinant protein of interest, wherein the amino acid sequence of the co-expressed protein has at least 90% homology to an amino acid sequence according to SEQ ID NO: 1, or wherein the amino acid sequence of the co-expressed protein has at least 90% sequence identity to an amino acid sequence according to SEQ ID NO: 2.

In a preferred embodiment of the second aspect of the present invention, the secreted recombinant protein of interest is expressed transiently, constitutively and/or inducibly/conditionally.

In another preferred embodiment of the second aspect of the present invention, the cell comprises a recombinant polynucleotide sequence encoding the secreted recombinant protein of interest, more preferably the cell comprises a non-genomic recombinant polynucleotide sequence, an expression cassette or a vector comprising the recombinant polynucleotide sequence encoding the secreted recombinant protein of interest.

In one preferred embodiment of the second aspect of the present invention, the expressed recombinant polynucleotide sequence in the cell encoding the secreted recombinant protein of interest comprises integrated CU-rich synonymous codons within the coding sequence, preferably wherein the recombinant polynucleotide sequence is an mRNA sequence.

In a preferred embodiment of the second aspect of the present invention, the cell is selected from the group consisting of CHO cells, BHK 21 cells, HEK293 cells, C127, A549, Sp2/0, YB2/0, SF-9 cells, NS0 cells, Vero cells, and any derivatives thereof.

In another preferred embodiment of the second aspect of the present invention, the secreted recombinant protein of interest is suitable for protein-based therapies, more preferably wherein the secreted recombinant protein is selected from the group consisting of an antibody, such as a therapeutic antibody, or an antigen-binding fragment thereof, or a nanobody, or an antibody-drug conjugate, or a recombinant fusion protein, or an exosome, or a cytokine, such as IFN-β, or a hormone, such as insulin, or a hormone analogue, such as a GnRH analogue.

According to the third aspect of the present invention, a use of the eukaryotic cell according to the second aspect of the present invention is provided in a method of producing a secreted recombinant protein of interest in vitro.

### DESCRIPTION OF FIGURES

FIG. 1 shows enhanced secretion of secreted proteins by overexpression of HDLBP/Vigilin (VgSEC platform) in comparison to standard processes for protein production

FIG. 2 shows SEAP and Gaussia luciferase activity measurements in parental HEK293 (WT) and cells stably transfected with piggybac transposon carrying a doxycycline-inducible HDLBP in the presence (+DOX) and absence (−DOX) of inducing amounts of doxycycline; SEAP and Gaussia luciferase signal was quantified in cell culture medium and normalized to the intracellular Firefly luciferase (Fluc) activity (experiment was carried out 5 times with at least 5 technical replicates).

FIG. 3 shows (A) Western analysis of wild type A549 cells (WT) and A549 cells stably transfected with a piggyback construct over-expressing HDLBP (OE), and (B) SEAP activity measurements in A459 wild type cells (WT), Vigilin/HDLBP knockout cells (KO), and Vigilin/HDLBP overexpressing cells (OE).

FIG. 4 shows (A) wild-type (WT) and codon-optimized versions of SEAP (opt 1, and opt 2), and (B) relative SEAP enzyme activity reflecting expression and secretion in HEK 293 WT and HDLBP-overexpressing cells expressing SEAP WT or codon-optimized SEAP proteins.

FIG. 5 shows the strategy for generation of CHO cell lines with inducible overexpression of HDLBP and EPO-FC, wherein the EPO-Fc Opt gene contains Leu, Ser and Pro codons that are more CU-rich.

FIG. 6 shows (A) a Western analysis of CHO cells (CHO cells WT) expressing only doxycycline (dox)-inducible EPO-Fc (EPO WT) and codon-optimized EPO-Fc (EPO Opt) genes and CHO cells with doxycycline-inducible HDLBP-overexpression construct (CHO cells HDLBP OE) expressing EPO WT and EPO Opt genes using anti-GAPDH, anti-HDLBP and anti-Fc antibodies; Ponceau staining as loading control, and (B) ELISA for EPO-Fc in the supernatant of CHO cells with no HDLBP overexpression (control) and HDLBP-overexpressing CHO cells (HDLBP OE) stably transfected with EPO-Fc (EPO-WT) and codon-optimized EPO-Fc construct (EPO Opt).

FIG. 7 shows (A) a Western analysis for EPO-Fc expression in untreated (control) CHO cells and cells treated with various compounds in the indicated concentrations; PHA-P—phytohemagglutinin; TSC—testosterone C; beta-ED—beta-estradiol; SPD—spermidine; CS—cholesterol, using anti-GAPDH, anti-HDLBP and anti-Fc antibodies; Ponceau staining as loading control, and (B) ELISA for EPO-Fc in the supernatant of untreated control cells and cells treated with various compounds in the indicated concentrations; PHA-P—phytohemagglutinin; TSC—testosterone C; beta-ED—beta-estradiol; SPD—spermidine; CS—cholesterol.

### DETAILED DESCRIPTION OF THE INVENTION

The present invention is based on the recognition that increased (co-)expression of HDLBP (High density lipoprotein-binding protein)/Vigilin in a eukaryotic cell enhances expression and secretion of secretory proteins from this cell, in particular in cellular expression systems for recombinant expression of secretory proteins. The present inventors intensively studied secretory pathways and successfully identified HDLBP/Vigilin and its important role for the efficiency of translation, expression and secretion of secretory proteins in eukaryotic cells.

HDLBP (also known as Vigilin; Vigilin and HDLBP may be used interchangeably herein) is a conserved and ubiquitously expressed RNA-binding protein localized both to the cytosol and the ER membrane. In humans, there are three major isoforms referred to as Isoform a (NCBI Reference Sequence NP_001307894.1; SEQ ID NO: 1), Isoform b (NCBI Reference Sequence NP_001230829.1; SEQ ID NO: 2) and Isoform c (NCBI Reference Sequence NP_001307896.1; SEQ ID NO: 3).

Homologous proteins are also known from D. melanogaster (Dodeca-satellite-binding protein 1, isoform A; NCBI Reference Sequence NP_995886.1; SEQ ID NO: 4), mouse (Vigilin, NCBI Reference Sequence NP_598569.1; SEQ ID NO: 5), Chinese hamster (Vigilin, NCBI Reference Sequence XP_027253465.1; SEQ ID NO: 6), green monkey (Vigilin Isoform X1, NCBI Reference Sequence XP_037856519.1; SEQ ID NO: 7), and other species. Any of these homologous proteins may be used in the context of the present invention, for example by co-expressing the respective homologue in an expression system derived from the same or a closely related organism.

The human isoform contains 15 hnRNP K-homology (KH) RNA-binding domains (RBDs). KH domains are high affinity RNA recognition elements (RREs), most commonly tetranucleotides as observed for FMRP27, SF1, HNRNPK and others. HDLBP and its yeast orthologue SCP160 have been found to contribute to many biological processes such as translation or protein aggregation, and have been linked to carcinogenesis.

Recently, HDLBP has been shown to be required for replication of flaviviruses ZIKV and DENV. HDLBP is also a promising target for cardiovascular research, since it appears to lead to less atherosclerotic plaques upon hepatic HDLBP knockdown in atherosclerosis prone Ldlr^{-/-}mice. However, functional aspects of HDLBP binding to RNA and mechanistic events during translation remain uncertain.

As part of the present invention, HDLBP binding sites were assayed in a transcriptome-wide manner by PAR-CLIP and their potential function as selective sequence determinants of ER-bound mRNAs was discovered. HDLBP directly and specifically interacted with a high percentage of at least 80% of all ER-localized mRNAs and was primarily bound to long CU-rich motifs in their coding sequence, a unique feature which is much more frequently found in membrane-bound compared to cytosolic mRNAs.

Biochemical, transcriptomic and proteomic methods were used to evaluate the functional consequences of HDLBP absence on ER translational efficiency, protein synthesis and secretion and highlighted its requirement for these biological processes.

Based on these considerations, the present inventors recognized the relevance and suitability of cellular expression systems overexpressing HDLBP for increased production of secreted proteins.

Accordingly, in a first aspect of the invention, a method is provided for producing a secreted recombinant protein of interest in a cell, wherein the method comprises artificially co-expressing a protein different from the secreted recombinant protein of interest, wherein the amino acid sequence of the co-expressed protein has at least 90% sequence identity to an amino acid sequence according to SEQ ID NO: 1, or wherein the amino acid sequence of the co-expressed protein has at least 90% sequence identity to an amino acid sequence according to SEQ ID NO: 2.

The term “producing” as it is used herein means the production of a recombinant protein using eukaryotic cells that are capable to produce this protein of interest by translation of a nucleotide sequence. As a “recombinant” polynucleotide or protein, a polynucleotide or protein may be described which has been introduced into the producing cell by means of transfection, transduction or other means of genetic engineering as commonly known in the art. Also, a “recombinant” polynucleotide or protein may be one that is not natively present in the producing cell.

For example, for producing a protein of interest, a recombinant polynucleotide sequence coding for one or more protein/s of interest can be introduced into the cell, which protein/s is/are then to be produced by said cell as part of the normal cellular transcription/translation process. Such nucleic acids can be introduced into the cell by various routes commonly known in the art.

Suitable vehicles of transport for the introduction of nucleic acids, which are to be transcribed and/or translated by the cell for protein production are, for example, vectors containing DNA. Also envisaged within the present invention are single-stranded nucleic acids such as RNA, preferably where the single-stranded nucleic acid to be introduced is mRNA. Alternatively, such nucleic acids coding for a protein of interest may be stably integrated (as single copy or multiple copies) into the genome of the eukaryotic cell used for protein production.

The eukaryotic cell used as expression system needs to take up the nucleic acid coding for the protein to be produced. This uptake may be mediated by various pathways of genetic transformation, which are well known to a skilled person in the art. Non-restrictive examples include electroporation, chemical-based transfection, particle-based transfection, injection and/or transduction of eukaryotic cells.

In principle, the method of the present invention can be applied in context of any expression system and for the production of any secreted protein of interest. Thus, an existing expression system for a secreted protein may be employed and modified to achieve overexpression of HDLBP in order to obtain the benefits of the present invention. These pathways of protein production can be carried out on any scale and are also suitable for generating large quantities of the protein of interest.

A “secreted protein” is to be understood as any protein, which is secreted by a cell, which means that the cell delivers the produced protein of interest to the outside of the cell or into the external medium.

A “recombinant protein of interest” may be any protein expressed by the cell characterized by the presence of a heterologous or recombinant polynucleotide sequence encoding for said protein within said cell.

In particular, while the coding sequence comprised in said recombinant polynucleotide sequence may be identical to a homologous coding sequence within the cell, other differences to homologous sequences within the cell are preferably present within the recombinant polynucleotide sequence, e.g. in regulatory regions of the polynucleotide sequence such as enhancer, promoter or other such regions.

Also, the recombinant polynucleotide sequence may comprise coding sequences that are heterologous and/or exogenous to the eukaryotic cell used as the expression system. In one embodiment, polycistronic expression systems may be employed which may preferably comprise vectors using internal ribosome entry sites (IRES) or 2A peptides.

Such elements are exemplarily described in Yeo J H M, et al. Methods Mol Biol. 2018; 1827:335-349. doi: 10.1007/978-1-4939-8648-4; Cruz T A et al. Biotechnol Lett 42, 2511-2522 (2020). https://doi.org/10.1007/s10529-020-02952-8, or Chng J et al. MAbs. 2015; 7(2):403-412. doi: 10.1080/19420862.2015.1008351, but are also well known in the field and to experts skilled in the art.

The terms “artificially” and “artificially co-expressing”, as they are used herein, are deemed to be understood as an expression of the co-expressed protein which does not take place naturally in the unmodified eukaryotic cell or expression system, in particular an increased or enhanced expression thereof.

Accordingly, artificial co-expression of the co-expressed protein according to the present invention preferably leads to higher levels of said protein within the eukaryotic cell in comparison to levels of said protein in native or unmodified eukaryotic cells. Thus, native cellular expression of the co-expressed protein different from the secreted recombinant protein of interest may nevertheless occur within said cell, independent of the artificial co-expression.

“Co-expressed” or “co-expression”, as used herein, is defined as simultaneous expression of two (or even more) different genes, in particular two or more different recombinant genes. The co-expressed protein of interest may be expressed within the cell transiently, constitutively and/or inducibly/conditionally, preferably the co-expressed protein is expressed constitutively.

According to the present invention, the co-expressed protein has at least 90% sequence identity to an amino acid sequence according to SEQ ID NO: 1, or wherein the amino acid sequence of the co-expressed protein has at least 90% sequence identity to an amino acid sequence according to SEQ ID NO: 2.

According to the present invention, the co-expressed protein may be a protein which is homologous to the protein referred to as Vigilin or HDLBP in humans. In one embodiment of the present invention, the co-expressed protein is the respective Vigilin/HDLBP homologue of the same species as the eukaryotic cell used as the expression system, or a protein having at least 90% sequence identity to the amino acid sequence of the respective Vigilin/HDLBP homologue.

According to one embodiment of the present invention, protein sequences form part of the invention as the co-expressed protein which consist of or comprise a protein sequence being at least 90% identical to the referenced protein sequences disclosed herein, preferably at least 95% identical, more preferably at least 98% identical, particularly preferably at least 99% identical.

The determination of percent identity between two sequences is accomplished according to the present invention by using the mathematical algorithm of Karlin and Altschul (Proc. Natl. Acad. Sci. USA (1993) 90: 5873-5877). Such an algorithm is the basis of the BLASTN and BLASTP programs of Altschul et al. (J. Mol. Biol. (1990) 215: 403-410). BLAST nucleotide searches are performed with the BLASTN program. To obtain gapped alignments for comparative purposes, Gapped BLAST is utilized as described by Altschul et al. (Nucleic Acids Res. (1997) 25: 3389-3402). When utilizing BLAST and Gapped BLAST programs, the default parameters of the respective programs are used.

According to one specific embodiment of the present invention, protein sequences forming part of the present invention as defined above by a given percent identity to the individualized sequences of Vigilin/HDLBP homologues are those that maintain the function of the respective Vigilin/HDLBP homologues.

In one embodiment, only those protein sequences are encompassed which are able to increase the production of a secreted recombinant protein of interest when co-expressed in the same cell, for example as measured according to the present disclosure.

In one embodiment, the protein sequence of the co-expressed protein may be the protein sequence of SEQ ID NO: 1. In another embodiment, the protein sequence of the co-expressed protein may be the protein sequence of SEQ ID NO: 2. In yet another embodiment, the protein sequence of the co-expressed protein may be the protein sequence of SEQ ID NO: 3.

In one embodiment, the protein sequence of the co-expressed protein may be the protein sequence of SEQ ID NO: 4. In one embodiment, the protein sequence of the co-expressed protein may be the protein sequence of SEQ ID NO: 5. In one embodiment, the protein sequence of the co-expressed protein may be the protein sequence of SEQ ID NO: 6. In one embodiment, the protein sequence of the co-expressed protein may be the protein sequence of SEQ ID NO: 7. In specific embodiments, the protein sequence of the co-expressed protein corresponds to the species of the expression system or eukaryotic cell used in the inventive method.

Accordingly, in one embodiment, the protein sequence of the co-expressed protein may be the protein sequence of SEQ ID NO: 1, SEQ ID NO: 2, or SEQ ID NO: 3, if a human cell is used, preferably HEK293 cells, derivatives thereof, or other cell lines of human origin.

In a different embodiment, the protein sequence of the co-expressed protein may be the protein sequence of SEQ ID NO: 4, if an insect cell is used, preferably Sf-9 cells, derivatives thereof, or other cell lines of insect origin.

In another embodiment, the protein sequence of the co-expressed protein may be the protein sequence of SEQ ID NO: 5 or 6, if a mouse, rat or hamster cell is used, preferably CHO cells, BHK 21 cells, C127 cells, SP2/0 cells, YB2/0 cells or NS0 cells.

In one embodiment, the protein sequence of the co-expressed protein may be the protein sequence of SEQ ID NO: 7, if a monkey cell is used, preferably Vero cells.

In view of the substantial homology of Vigilin/HDLPBP in different species, it may also be preferred to use any of the proteins with protein sequences of SEQ ID NO: 1, 2, 3, 5, 6 and 7 in any eukaryotic cell of mammalian origin.

In one preferred embodiment, the co-expressed protein consists of any of the Vigilin/HDLBP homologue sequences disclosed herein, more preferably the co-expressed protein consists of the amino acid sequence of SEQ ID NO: 1.

In the following, the amino acid sequences of the specific Vigilin/HDLBP homologues are given in the common one-letter code:

(human Isoform a)
>NP_001307894 HDLBP (Homo sapiens) Isoform a
SEQ ID NO: 1
MSSVAVLTQESFAEHRSGLVPQQIKVATLNSEEESDPPTYKDAFPPLPEK
AACLESAQEPAGAWGNKIRPIKASVITQVFHVPLEERKYKDMNQFGEGEQ
AKICLEIMQRTGAHLELSLAKDQGLSIMVSGKLDAVMKARKDIVARLQTQ
ASATVAIPKEHHRFVIGKNGEKLQDLELKTATKIQIPRPDDPSNQIKITG
TKEGIEKARHEVLLISAEQDKRAVERLEVEKAFHPFIAGPYNRLVGEIMQ
ETGTRINIPPPSVNRTEIVFTGEKEQLAQAVARIKKIYEEKKKKTTTIAV
EVKKSQHKYVIGPKGNSLQEILERTGVSVEIPPSDSISETVILRGEPEKL
GQALTEVYAKANSFTVSSVAAPSWLHRFIIGKKGQNLAKITQQMPKVHIE
FTEGEDKITLEGPTEDVNVAQEQIEGMVKDLINRMDYVEINIDHKFHRHL
IGKSGANINRIKDQYKVSVRIPPDSEKSNLIRIEGDPQGVQQAKRELLEL
ASRMENERTKDLIIEQRFHRTIIGQKGERIREIRDKFPEVIINFPDPAQK
SDIVQLRGPKNEVEKCTKYMQKMVADLVENSYSISVPIFKQFHKNIIGKG
GANIKKIREESNTKIDLPAENSNSETIIITGKRANCEAARSRILSIQKDL
ANIAEVEVSIPAKLHNSLIGTKGRLIRSIMEECGGVHIHFPVEGSGSDTV
VIRGPSSDVEKAKKQLLHLAEEKQTKSFTVDIRAKPEYHKFLIGKGGGKI
RKVRDSTGARVIFPAAEDKDQDLITIIGKEDAVREAQKELEALIQNLDNV
VEDSMLVDPKHHRHFVIRRGQVLREIAEEYGGVMVSFPRSGTQSDKVTLK
GAKDCVEAAKKRIQEIIEDLEAQVTLECAIPQKFHRSVMGPKGSRIQQIT
RDFSVQIKFPDREENAVHSTEPVVQENGDEAGEGREAKDCDPGSPRRCDI
IIISGRKEKCEAAKEALEALVPVTIEVEVPFDLHRYVIGQKGSGIRKMMD
EFEVNIHVPAPELQSDIIAITGLAANLDRAKAGLLERVKELQAEQEDRAL
RSFKLSVTVDPKYHPKIIGRKGAVITQIRLEHDVNIQFPDKDDGNQPQDQ
ITITGYEKNTEAARDAILRIVGELEQMVSEDVPLDHRVHARIIGARGKAI
RKIMDEFKVDIRFPQSGAPDPNCVTVTGLPENVEEAIDHILNLEEEYLAD
VVDSEALQVYMKPPAHEEAKAPSRGFVVRDAPWTASSSEKAPDMSSSEEF
PSFGAQVAPKTLPWGPKR
(human Isoform b)
>NP_001230829 HDLBP (Homo sapiens) Isoform b
SEQ ID NO: 2
MHLAERDRWLFVATVMMHFVSIKSGFPGLCVGVRSTMSSVAVLTQESFAE
HRSGLVPQQIKVATLNSEEESDPPTYKDAFPPLPEKAACLESAQEPAGAW
GNKIRPIKASVITQVFHVPLEERKYKDMNQFGEGEQAKICLEIMQRTGAH
LELSLAKDQGLSIMVSGKLDAVMKARKDIVARLQTQASATVAIPKEHHRF
VIGKNGEKLQDLELKTATKIQIPRPDDPSNQIKITGTKEGIEKARHEVLL
ISAEQDKRAVERLEVEKAFHPFIAGPYNRLVGEIMQETGTRINIPPPSVN
RTEIVFTGEKEQLAQAVARIKKIYEEKANSFTVSSVAAPSWLHRFIIGKK
GQNLAKITQQMPKVHIEFTEGEDKITLEGPTEDVNVAQEQIEGMVKDLIN
RMDYVEINIDHKFHRHLIGKSGANINRIKDQYKVSVRIPPDSEKSNLIRI
EGDPQGVQQAKRELLELASRMENERTKDLIIEQRFHRTIIGQKGERIREI
RDKFPEVIINFPDPAQKSDIVQLRGPKNEVEKCTKYMQKMVADLVENSYS
ISVPIFKQFHKNIIGKGGANIKKIREESNTKIDLPAENSNSETIIITGKR
ANCEAARSRILSIQKDLANIAEVEVSIPAKLHNSLIGTKGRLIRSIMEEC
GGVHIHFPVEGSGSDTVVIRGPSSDVEKAKKQLLHLAEEKQTKSFTVDIR
AKPEYHKFLIGKGGGKIRKVRDSTGARVIFPAAEDKDQDLITIIGKEDAV
REAQKELEALIQNLDNVVEDSMLVDPKHHRHFVIRRGQVLREIAEEYGGV
MVSFPRSGTQSDKVTLKGAKDCVEAAKKRIQEIIEDLEAQVTLECAIPQK
FHRSVMGPKGSRIQQITRDFSVQIKFPDREENAVHSTEPVVQENGDEAGE
GREAKDCDPGSPRRCDIIIISGRKEKCEAAKEALEALVPVTIEVEVPFDL
HRYVIGQKGSGIRKMMDEFEVNIHVPAPELQSDIIAITGLAANLDRAKAG
LLERVKELQAEQEDRALRSFKLSVTVDPKYHPKIIGRKGAVITQIRLEHD
VNIQFPDKDDGNQPQDQITITGYEKNTEAARDAILRIVGELEQMVSEDVP
LDHRVHARIIGARGKAIRKIMDEFKVDIRFPQSGAPDPNCVTVTGLPENV
EEAIDHILNLEEEYLADVVDSEALQVYMKPPAHEEAKAPSRGFVVRDAPW
TASSSEKAPDMSSSEEFPSFGAQVAPKTLPWGPKR
(human Isoform c)
>NP_001307896 HDLBP (Homo sapiens) Isoform c
SEQ ID NO: 3
MSSVAVLTQESFAEHRSGLVPQQIKVATLNSEEESDPPTYKDAFPPLPEK
AACLESAQEPAGAWGNKIRPIKASVITQVFHVPLEERKYKDMNQFGEGEQ
AKICLEIMQRTGAHLELSLAKDQGLSIMVSGKLDAVMKARKDIVARLQTQ
ASATVAIPKEHHRFVIGKNGEKLQDLELKTATKIQIPRPDDPSNQIKITG
TKEGIEKARHEVLLISAEQDKRAVERLEVEKAFHPFIAGPYNRLVGEIMQ
ETGTRINIPPPSVNRTEIVFTGEKEQLAQAVARIKKIYEEKKKKTTTIAV
EVKKSQHKYVIGPKGNSLQEILERTGVSVEIPPSDSISETVILRGEPEKL
GQALTEVYAKANSFTVSSVAAPSWLHRFIIGKKGQNLAKITQQMPKVHIE
FTEGEDKITLEGPTEDVNVAQEQIEGMVKDLINRMDYVEINIDHKFHRHL
IGKSGANINRIKDQYKVSVRIPPDSEKSNLIRIEGDPQGVQQAKRELLEL
ASRMENERTKDLIIEQRFHRTIIGQKGERIREIRDKFPEVIINFPDPAQK
SDIVQLRGPKNEVEKCTKYMQKMVADLVENSYSISVPIFKQFHKNIIGKG
GANIKKIREESNTKIDLPAENSNSETIIITGKRANCEAARSRILSIQKDL
ANIAEVEVSIPAKLHNSLIGTKGRLIRSIMEECGGVHIHFPVEGSGSDTV
VIRGPSSDVEKAKKQLLHLAEEKQTKSFTVDIRAKPEYHKFLIGKGGGKI
RKVRDSTGARVIFPAAEDKDQDLITIIGKEDAVREAQKELEALIQNLDNV
VEDSMLVDPKHHRHFVIRRGQVLREIAEEYGGVMVSFPRSGTQSDKVTLK
GAKDCVEAAKKRIQEIIEDLEAQVTLECAIPQKFHRSVMGPKGSRIQQIT
RDFSVQIKFPDREENAVHSTEPVVQENGDEAGEGREAKDCDPGSPRRCDI
IIISGRKEKCEAAKEALEALVPVTIEVEVPFDLHRYVIGQKGSGIRKMMD
EFEVNIHVPAPELQSDIIAITGLAANLDRAKAGLLERVKELQAEQEDRAL
RSFKLSVTVDPKYHPKIIGRKGAVITQIRLEHDVNIQFPDKDDGNQPQDQ
ITITGYEKNTEAARDAILRIVGELEQMVSEDVPLDHRVHARIIGARGKAI
RKIMDEFKVDIRFPQSGAPDPNCVTVTGLPENVEEAIDHILNLEEEYLAD
VVDSEALQVYMKPPAHEEAKAPSRGFVVRDAPWTASSSEKGRDDGMGPRA
(Dp1 from D. melanogaster)
>NP_995886.1 Dodeca-satellite-binding
protein 1, isoform A [Drosophila
melanogaster]
SEQ ID NO: 4
MQAAAVMEETNNATTIEQQPIALINGQEQVANEQQPSSPTSVATPTSTTS
GGTGNATPAFSYDDLFPALPANTSAQSQSGASGSTLARVTSSQKTHIVHV
PCKERKSTESEKFGEGESKRICQQITKETGAQIEIVSGKNQSLTFLIKGK
QSELLDARRKILMGFSTQASRQVTVPREHFRVILGKGGQRLREIERVTAT
RINIPSQSDESEFITIAGTKEGIAQAEQEIRQLSAEQYKKSSDRITVPKV
YHPFIVGPYSENLNKLQEETGARINVPPQQVQKDEIVISGEKDAVAAAKA
KVEAIYKDMEKKCSTVSVEVAKPKHRYVIGPKGSTIAEILQLTGVSVEMP
PNDSPSETITLRGPQVALGNALTVVYQKSNSVKSVEINAAHWIHKYVIGR
KGANMKQLEEDCPNVNVNCLEDKIKLEGDPENVDRAVAYLSEIIKNYEEN
FTFEVMTVNPSYYKHIIGKAGANVNRLKDELKVNINIEEREGQNNIRIEG
PKEGVRQAQLELQEKIDKLENEKSKDVIIDRRLHRSIIGAKGEKIREVKD
RYRQVTITIPTPQENTDIVKLRGPKEDVDKCHKDLLKLVKEIQESSHIIE
VPIFKQFHKFVIGKGGANIKKIRDETQTKIDLPAEGDTNEVIVITGKKEN
VLEAKERIQKIQNELSDIVTEEVQIPPKYYNSIIGTGGKLISSIMEECGG
VSIKFPNSDSKSDKVTIRGPKDDVEKAKVQLLELANERQLASFTAEVRAK
QQHHKFLIGKNGASIRKIRDATGARIIFPSNEDTDKEVITIIGKEESVKK
AREQLEAIIKECDEVTEGEVSVDPKHHKHFVAKRGFILHRISEECGGVMI
SFPRVGINSDKVTIKGAKDCIEAARQRIEEIVADLEAQTTIEVVIPQRHH
RTIMGARGFKVQQVTFEFDVQIKFPDRDATEPVEGLTNGGSGENGGENEG
QEGEQEVEKEAEQEPVRQCDVIRITGRIEKCEAAKQALLDLIPIEEELSV
PFDLHRTIIGPRGANVRQFMSKHDVHVELPPSELKSDVIKVCGTPARVAE
AREALVKMIEDYEADRADRELRSFVLQVDVDTEFHSKLIGRHGAVINKLR
ADHDVIISLPKRDEPNDRIISITGYQANAEAARDAILEIVGDPETLHREV
IEIDKRIHPHLIGQRRRTIRKIIEDNKVNIKFSADDDNPNSIFISGKIED
VENVKELLFGMAEDYERDYLDNVAIAPPTIGAFLTGQGSGSDAAGASENG
FVIKDAPWEKQKQAKNLTAPNTQSQEDFPHFAAGGAPVASTPITSVWGPK
N
(Vigilin from M. musculus)
>NP_598569.1 vigilin [Mus musculus]
SEQ ID NO: 5
MSSVAVLTQESFAEHRSGLVPQQIKVATLNSEEENDPPTYKDAFPPLPEK
AACLESAQEPAGAWSNKIRPIKASVITQVFHVPLEERKYKDMNQFGEGEQ
AKICLEIMQRTGAHLELSLAKDQGLSIMVSGKLDAVMKARKDIVARLQTQ
ASATVPIPKEHHRFVIGKNGEKLQDLELKTATKIQIPRPDDPSNQIKITG
TKEGIEKARHEVLLISAEQDKRAVERLEVEKAFHPFIAGPYNRLVGEIMQ
ETGTRINIPPPSVNRTEIVFTGEKEQLAQAVARIKKIYEEKKKKTTTIAV
EVKKSQHKYVIGPKGNSLQEILERTGVSVEIPPSDSISETVILRGEPEKL
GQALTEVYAKANSFTVSSVSAPSWLHRFIIGKKGQNLAKITQQMPKVHIE
FTEGEDKITLEGPTEDVNVAQEQIEGMVKDLINRMDYVEINIDHKFHRHL
IGKSGANINRIKDQYKVSVRIPPDSEKSNLIRIEGDPQGVQQAKRELLEL
ASRMENERTKDLIIEQRFHRTIIGQKGERIREIRDKFPEVIINFPDPAQK
SDIVQLRGPKNEVEKCTKYMQKMVADLVENSYSISVPIFKQFHKNIIGKG
GANIKKIREESNTKIDLPAENSNSETIIITGKRANCEAARSRILSIQKDL
ANIAEVEVSIPAKLHNSLIGTKGRLIRSIMEECGGVHIHFPVEGSGSDTV
VIRGPSSDVEKAKKQLLHLAEEKQTKSFTVDIRAKPEYHKFLIGKGGGKI
RKVRDSTGARIIFPAAEDKDQDLITIIGKEDAVREAQKELEALIQNLENV
VEDYMLVDPKHHRHFVIRRGQVLREIAEEYGGVMVSFPRSGTQSDKVTLK
GAKDCVEAAKKRIQEIIEDLEAQVTVECAIPQKFHRSVMGPKGSRIQQIT
RDYNVQIKFPDREENPVHSVEPSIQENGDEAGEGREAKETDPGSPRRCDI
IIISGRKEKCEAAKEALEALVPVTIEVEVPFDLHRYIIGQKGSGIRKMMD
EFEVNIHVPAPELQSDTIAITGLAANLDRAKAGLLDRVKELQAEQEDRAL
RSFKLSVTVDPKYHPKIIGRKGAVITQIRLEHEVNIQFPDKDDGNQPQDQ
ITITGYEKNTEAARDAILKIVGELEQMVSEDVPLDHRVHARIIGARGKAI
RKIMDEFKVDIRFPQSGAPDPNCVTVTGLPENVEEAIDHILNLEEEYLAD
VVDSEALQVYMKPPAHEESRAPSKGFVVRDAPWTSNSSEKAPDMSSSEEF
PSFGAQVAPKTLPWGPKR
(Vigilin from C. griseus)
>XP_027253465.1 vigilin [Cricetulus griseus]
SEQ ID NO: 6
MSSVAVLTQESFAEHRSGLVPQQIKVATLNSEEESDPPTYKDAFPPLPEK
AACLESAQEPAGAWSNKIRPIKASVITQVFHVPLEERKYKDMNQFGEGEQ
AKICLEIMQRTGAHLELSLAKDQGLSIMVSGKLDAVMKARKDIVARLQTQ
ASATVTIPKEHHRFVIGKNGEKLQDLELKTATKIQIPRPDDPSNQIKITG
TKEGIEKARHEVLLISAEQDKRAVERLEVEKAFHPFIAGPYNRLVGEIMQ
ETGTRINIPPPSVNRTEIVFTGEKEQLAQAVARIKKIYEEKKKKTTTIAV
EVKKSQHKYVIGPKGNSLQEILERTGVSVEIPPSDSISETVILRGEPEKL
GQALTEVYAKANSFTVSSVSAPSWLHRFIIGKKGQNLAKITQQMPKVHIE
FTEGEDKITLEGPTEDVSVAQEQIEGMVKDLINRMDYVEINIDHKFHRHL
IGKSGANINRIKDQYKVSVRIPPDSEKSNLIRIEGDPQGVQQAKRELLEL
ASRMENERTKDLIIEQRFHRTIIGQKGERIREIRDKFPEVIINFPDPAQK
SDIVQLRGPKNEVEKCTKYMQKMVADLVENSYSISVPIFKQFHKNIIGKG
GANIKKIREESNTKIDLPAENSNSETIVITGKRANCEAARSRILSIQKDL
ANIAEVEVSIPAKLHNSLIGTKGRLIRSIMEECGGVHIHFPVEGSGSDTV
VIRGPSSDVEKAKKQLLHLAEEKQTKSFTVDIRAKPEYHKFLIGKGGGKI
RKVRDSTGARIIFPAAEDKEQDLITIVGKEDAVREAQKELEALIQNLDNV
VEDYMLVDPKHHRHFVIRRGQVLREIAEEYGGVMVSFPRSGTQSDKVTLK
GAKDCVEAAKKRIQEIIEDLEAQVTVECAIPQKFHRSVMGPKGSRIQQIT
RDYNVQIKFPDREENPVHSVEPSIQENGDEAGEGREAKETDPGSPRRCDI
IIISGRREKCEAAKEALEALVPVTIEVEVPFDLHRYVIGQKGSGIRKMMD
EFEVNIHVPAPELQSDIIAITGLVANLDRAKTGLLDRVKELQAEQEDRAL
RSFKLSVTVDPKYHPKIIGRKGAVITQIRLEHDVNIQFPDKDDGNQPQDQ
ITITGYEKNTEAARDAILKIVGELEQMVSEDVPLDHRVHARIIGARGKAI
RKIMDEFKVDIRFPQTGAPDPNCVTVTGLPENVEEAIDHILNLEEEYLAD
VVDSEALQVYMKPPAHEESKAPSKGFVVRDAPWTSNSSEKAPDMSSSEEF
PSFGAQVAPKTLPWGPKR
(Vigilin from C. sabaeus)
>XP_037856519.1 vigilin isoform X1
[Chlorocebus sabaeus]
SEQ ID NO: 7
MSSVAVLTQESFAEHRSGLVPQQIKEREDSTERMHPPVVSALSVATLNSE
EESDPPTYKDAFPPLPEKAACLESAQEPAGAWGNKIRPIKASVITQVFHV
PLEERKYKDMNQFGEGEQAKICLEIMQRTGAHLELSLAKDQGLSIMVSGK
LDAVMKARKDIVARLQTQASATVAIPKEHHRFVIGKNGEKLQDLELKTAT
KIQIPRPDDPSNQIKITGTKEGIEKARHEVLLISAEQDKRAVERLEVEKA
FHPFIAGPYNRLVGEIMQETGTRINIPPPSVNRTEIVFTGEKEQLAQAVA
RIKKIYEEKKKKTTTIAVEVKKSQHKYVIGPKGNSLQEILERTGVSVEIP
PSDSISETVILRGEPEKLGQALTEVYAKANSFTVSSVAAPSWLHRFIIGK
KGQNLAKITQQMPKVHIEFTEGEDKITLEGPTEDVNVAQEQIEGMVKDLI
NRMDYVEINIDHKFHRHLIGKSGANINRIKDQYKVSVRIPPDSEKSNLIR
IEGDPQGVQQAKRELLELASRMENERTKDLIIEQRFHRTIIGQKGERIRE
IRDKFPEVIINFPDPAQKSDIVQLRGPKNEVEKCTKYMQKMVADLVENSY
SISVPIFKQFHKNIIGKGGANIKKIREESNTKIDLPAENSNSETIIITGK
RANCEAARSRILSIQKDLANIAEVEVSIPAKLHNSLIGTKGRLIRSIMEE
CGGVHIHFPVEGSGSDTVVIRGPSSDVEKAKKQLLHLAEEKQTKSFTVDI
RAKPEYHKFLIGKGGGKIRKVRDSTGARVIFPAAEDKDQDLITIIGKEDA
VREAQKELEALIQNLDNVVEDSMLVDPKHHRHFVIRRGQVLREIAEEYGG
VMVSFPRSGTQSDKVTLKGAKDCVEAAKKRIQEIIEDLEAQVTLECAIPQ
KFHRSVMGPKGSRIQQITRDFNVQIKFPDREENPVHSVEPVVQENGDEAG
EGREAKDSDPGSPRRCDIIIISGRKEKCEAAKEALEALVPVTIEVEVPFD
LHRYVIGQKGSGIRKMMDEFEVNIHVPAPELQSDIIAITGLAANLDRAKA
GLLERVKELQAEQEDRALRSFKLSVTVDPKYHPKIIGRKGAVITQIRLEH
DVNIQFPDKDDGNQPQDQITITGYEKNTEAARDAILRIVGELEQMVSEDV
PLDHRVHARIIGARGKAIRKIMDEFKVDIRFPQSGAPDPNCVTVTGLPEN
VEEAIDHILNLEEEYLADVVDSEVLQVYMKPPAHEEAKAPSRGFVVRDAP
WTASSSEKAPDMSSSEEFPSFGAQVAPKTLPWGPKR

According to the present invention, the secreted recombinant protein of interest may be any protein which is targeted to the secretory pathway. In one embodiment, the secreted recombinant protein of interest is a proteinaceous compound that is suitable for a therapeutic use. In a specific embodiment, the secreted recombinant protein is selected from the group consisting of an antibody, such as a therapeutic antibody, or an antigen-binding fragment thereof, or a cytokine, such as IFN-β, or a hormone, such as insulin, or a hormone analogue, such as a GnRH analogue.

“Cytokines” as used herein include all classes of cytokines, such as interferons, interleukins, colony-stimulating factors, tumor necrosis factors and chemokines.

Non-limiting examples for interferons include e.g. IFN-alpha (IFN-alpha-2a and IFN-alpha-2b), IFN-beta (IFN-beta-1a and IFN-beta-1b) and IFN-gamma. Non-limiting examples for interleukins include e.g. IL-1, IL-2, IL-3, IL-4, IL-5, IL-6, IL-7, IL-8, IL-9, IL-10, IL-11, IL-12, IL-13, IL-16, IL-18, and IL-23. Non-limiting examples for colony-stimulating factors include e.g. erythropoietin (EPO), thrombopoietin (TPO), G-CSF (granulocyte-colony stimulating factor), GM-CSF (granulocyte macrophage colony-stimulating factor) and M-CSF (macrophage colony stimulating factor). Non-limiting examples for tumor necrosis factors include e.g. TNF-alpha and TNF-beta.

Non-limiting examples for hormones include peptide hormones (proteohormones) and glycoprotein hormones.

Peptide hormones include Adiponectin, Adiuretin (vasopressin, ADH), Adrenomedullin, Agouti-Related Peptide (AGRP), Angiotensin II (AII), Anti-Müllerian hormone (AMH), Atrial Natriuretic Peptide (ANP), Bombesin, B-type natriuretic peptide (BNP), Calcitonin (CT), Cholecystokinin (CCK), CRH, C-type natriuretic peptide (CNP), Enteroglucagon (GLI), Erythropoietin (EPO), FGF19, FGF21, FGF23, Gastrin, Ghrelin, GHRH, Gastroinhibitory peptide (GIP), Glucagon, GnRH, Hepcidin, HGH (GH, STH), Homeostatic thymus hormone (HTH), IGF 1, IGF 2, Inhibin, Insulin, Leptin, MCH, Melatonin, Motilin, Neuropeptide Y (NPY), Neurotensin, Parathyroid hormone (PTH), Parathyroid hormone-related peptide (PTHrP), Placental growth hormone (hGH-V), Omentin, Osteocalcin, Oxytocin, Pancreatic polypeptide (PP), Peptide YY (PYY), Prolactin (PRL), Pro-opio-melanocortin derivatives (such as Alpha-MSH, Beta-MSH, Gamma-MSH, CLIP, Endorphins, Enkephalins, Corticotropin (ACTH), β-Lipotropin), Relaxin, Resistin, Secretin, Somatostatin, Substance P, Thymosin (Thymosin alpha-1 and Thymosin beta-4), Thymulin, Vasoactive intestinal peptide (VIP), Vaspin and Visfatin.

Glycoprotein hormones include, but are not limited to those, e.g. FSH, HCG (Beta-HCG), LH, Thyrostimulin (TSH 2) and TSH (TSH 1).

As used herein the term “hormone analogue” is deemed to be understood as substances that have a similar chemical (“analogue”) structure to the corresponding hormone and can therefore bind to the corresponding receptors and achieve the same effect as the corresponding hormone. Non-limiting examples for hormone analogues are e.g. GnRH analogue (gonadotropin releasing hormone).

One particular example for a secreted recombinant protein of interest as used within the present invention is EPO-Fc, a fusion protein between erythropoietin and the Fc (fragment crystallizable) domain of an antibody. As such, EPO-Fc is a representative example for expression of a cytokine as well as of an immunoglobulin-based therapeutic or an antibody-drug conjugate.

In order to assess the effects of the present invention on the expression and secretion of proteins of interest such as therapeutic immunoglobulins, cytokines, or the like, cells have first been transfected with an HDLBP overexpressing construct.

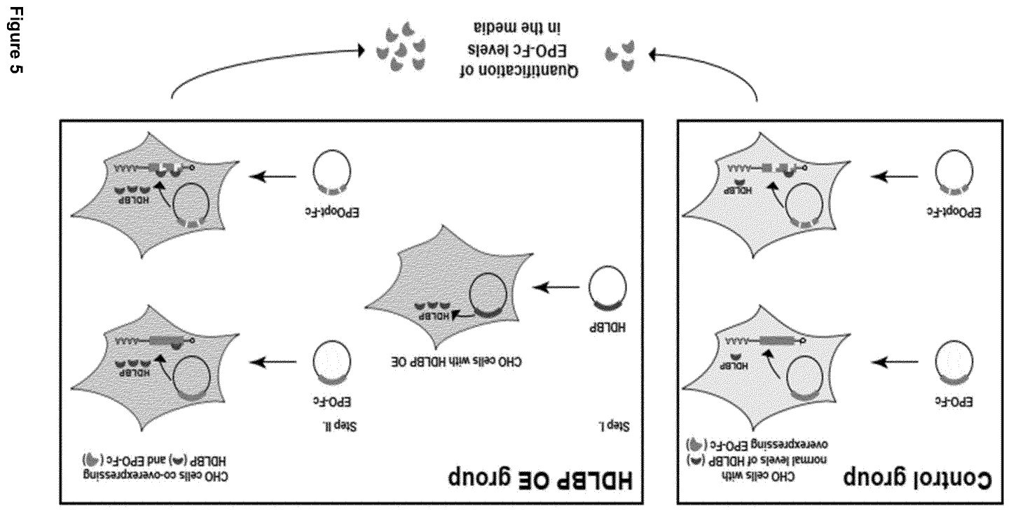

In a next step, the HDLBP overexpressing cells have been further transfected with either of an EPO-Fc WT construct (SEQ ID NO: 8 for the nucleotide sequence encoding EPO-Fc WT) or an EPO-Fc Opt construct (wherein the sequence has been codon-optimized by rendering Leu, Ser and Pro codons more CU-rich; in this particular sequence, 16 Pro codons, 20 Leu codons, and 7 Ser codons have been optimized accordingly; SEQ ID NO: 9 for the nucleotide sequence encoding EPO-Fc Opt). The strategy for establishing these cells is illustrated in FIG. 5.

In the following, the nucleotide sequences of the EPO-Fc WT construct and the EPO-Fc Opt construct are given in the common one-letter code, wherein the optimized codons can be recognized:

(EPO-Fc WT construct)
SEQ ID NO: 8
ATGGGGGTGCACGAATGTCCTGCCTGGCTGTGGCTTCTCCTGTCCCTGCT
GTCGCTCCCTCTGGGCCTCCCAGTCCTGGGCGCCCCACCACGCCTCATCT
GTGACAGCCGAGTCCTGGAGAGGTACCTCTTGGAGGCCAAGGAGGCCGAG
AATATCACGACGGGCTGTGCTGAACACTGCAGCTTGAATGAGAATATCAC
TGTCCCAGACACCAAAGTTAATTTCTATGCCTGGAAGAGGATGGAGGTCG
GGCAGCAGGCCGTAGAAGTCTGGCAGGGCCTGGCCCTGCTGTCGGAAGCT
GTCCTGCGGGGCCAGGCCCTGTTGGTCAACTCTTCCCAGCCGTGGGAGCC
CCTGCAGCTGCATGTGGATAAAGCCGTCAGTGGCCTTCGCAGCCTCACCA
CTCTGCTTCGGGCTCTGCGAGCCCAGAAGGAAGCCATCTCCCCTCCAGAT
GCGGCCTCAGCTGCTCCACTCCGAACAATCACTGCTGACACTTTCCGCAA
ACTCTTCCGAGTCTACTCCAATTTCCTCCGGGGAAAGCTGAAGCTGTACA
CAGGGGAGGCCTGCAGGACAGGGGACAGAGTTGAGCCCAAATCTGGTGAC
AAAACTAGTACATGCCCACCGTGCCCAGCACCTGAACTCCTGGGGGGACC
GTCAGTCTTCCTCTTCCCCCCAAAACCCAAGGACACCCTCATGATCTCCC
GGACCCCTGAGGTCACATGCGTGGTGGTGGACGTGAGCCACGAAGACCCT
GAGGTCAAGTTCAACTGGTACGTGGACGGCGTGGAGGTGCATAATGCCAA
GACAAAGCCGCGGGAGGAGCAGTACAACAGCACGTACCGTGTGGTCAGCG
TCCTCACCGTCCTGCACCAGGACTGGCTGAATGGCAAGGAGTACAAGTGC
AAGGTCTCCAACAAAGCCCTCCCAGCCCCCATCGAGAAAACCATCTCCAA
AGCCAAAGGGCAGCCCCGAGAACCACAGGTGTACACCCTGCCCCCATCCC
GGGATGAGCTGACCAAGAACCAGGTCAGCCTGACCTGCCTGGTCAAAGGC
TTCTATCCCAGCGACATCGCCGTGGAGTGGGAGAGCAATGGGCAGCCGGA
GAACAACTACAAGACCACGCCTCCCGTGCTGGACTCCGACGGCCCCTTCT
TCCTCTACAGCAAGCTCACCGTGGACAAGAGCAGGTGGCAGCAGGGGAAC
GTCTTCTCATGCTCCGTGATGCATGAGGCTCTGCACAACCACTACACGCA
GAAGAGCCTCTCCCTGTCTCCGGGTAAATAA
(EPO-Fc Opt construct)
SEQ ID NO: 9
ATGGGGGTGCACGAATGTCCTGCCTGGCTGTGGCTTCTCCTGTCCCTGCT
GTCGCTCCCTCTGGGCCTCCCAGTCCTGGGCGCCCCACCACGCCTCATCT
GTGACAGCCGAGTCCTGGAGAGGTACCTCTTGGAGGCCAAGGAGGCCGAG
AATATCACGACGGGCTGTGCTGAACACTGCAGCTTGAATGAGAATATCAC
TGTCCCTGACACCAAAGTTAATTTCTATGCCTGGAAGAGGATGGAGGTCG
GGCAGCAGGCCGTAGAAGTCTGGCAGGGCCTTGCCCTTCTGTCTGAAGCT
GTCCTGCGGGGCCAGGCCCTTCTTGTCAACTCTTCCCAGCCTTGGGAGCC
TCTTCAGCTTCATGTGGATAAAGCCGTCTCTGGCCTTCGCAGCCTCACCA
CTCTTCTTCGGGCTCTTCGAGCCCAGAAGGAAGCCATCTCCCCTCCTGAT
GCGGCCTCAGCTGCTCCTCTCCGAACAATCACTGCTGACACTTTCCGCAA
ACTCTTCCGAGTCTACTCCAATTTCCTCCGGGGAAAGCTTAAGCTGTACA
CAGGGGAGGCCTGCAGGACAGGGGACAGAGTTGAGCCCAAATCTGGTGAC
AAAACTAGTACATGCCCTCCTTGCCCAGCACCTGAACTTCTTGGGGGACC
TTCAGTCTTCCTCTTCCCTCCTAAACCCAAGGACACCCTCATGATCTCCC
GGACCCCTGAGGTCACATGCGTGGTGGTGGACGTGAGCCACGAAGACCCT
GAGGTCAAGTTCAACTGGTACGTGGACGGCGTGGAGGTGCATAATGCCAA
GACAAAGCCTCGGGAGGAGCAGTACAACTCTACGTACCGTGTGGTCTCTG
TCCTTACCGTCCTTCACCAGGACTGGCTTAATGGCAAGGAGTACAAGTGC
AAGGTCTCCAACAAAGCCCTTCCTGCCCCTATCGAGAAAACCATCTCCAA
AGCCAAAGGGCAGCCCCGAGAACCACAGGTGTACACCCTTCCTCCTTCCC
GGGATGAGCTTACCAAGAACCAGGTCTCTCTTACCTGCCTTGTCAAAGGC
TTCTATCCCAGCGACATCGCCGTGGAGTGGGAGAGCAATGGGCAGCCGGA
GAACAACTACAAGACCACGCCTCCTGTGCTTGACTCCGACGGCCCCTTCT
TCCTTTACTCTAAGCTCACCGTGGACAAGAGCAGGTGGCAGCAGGGGAAC
GTCTTCTCATGCTCCGTGATGCATGAGGCTCTGCACAACCACTACACGCA
GAAGTCTCTCTCCCTTTCTCCTGGTAAATAA

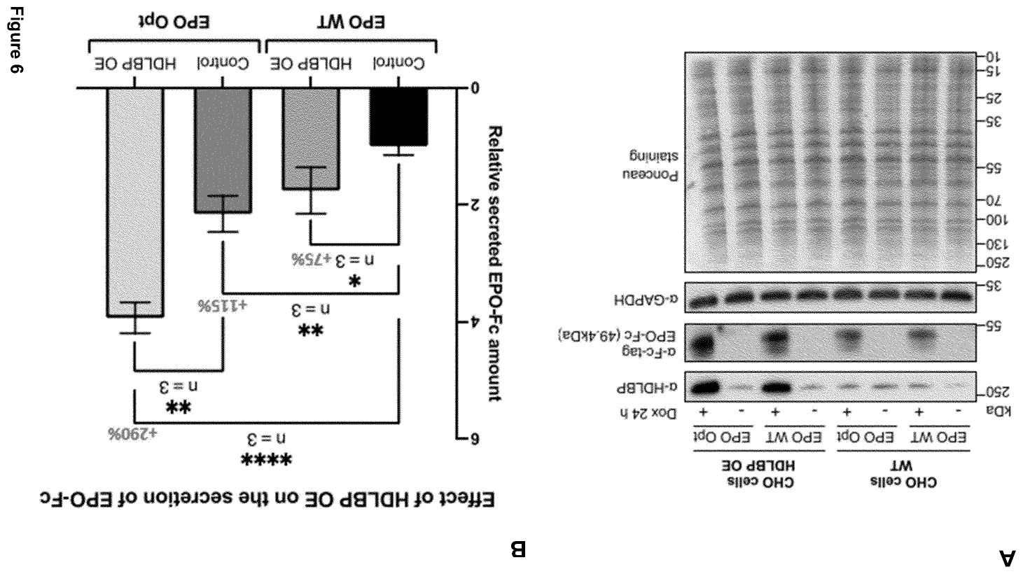

An analysis of the amounts of expressed EPO-Fc shows that the overexpression of HDLBP together with EPO-Fc WT already leads to an increase in protein expression and secretion by more than 75% over the control which does not overexpress HDLBP (cf. FIG. 6B, left part).

Strikingly, the use of a codon-optimized version of EPO-Fc which has a higher number of CU-rich codons already leads to an increase in protein expression and secretion of about 115% even in absence of HDLBP overexpression. It appears that this increase is caused already by the codon-optimization alone which has a striking effect on HDLBP naturally present in the cell.

The combination of codon optimization and HDLBP overexpression then leads to an increase by more than 290% over the control which does not overexpress HDLBP and does not use a codon optimized construct (cf. FIG. 6B, right part).

The production of proteins involving translation inevitably requires the provision of producing cells or cell lines. In principle, all cell lines which can be used for an artificial expression of a recombinant specific protein are suitable for this purpose. In one embodiment of the present invention, the cell is a eukaryotic cell.

Eukaryotic cells include, but are not limited to those, such as hamster cell lines (CHO and their derivatives), mouse cell lines (such as C127, NS0, SP2/0, YB2/0, XB2/09 and derivatives of all of them), or human cell lines (such as HEK293 and their derivatives, HT-1080, PER. C6, or Huh-7). Also included are cell lines from monkeys, such as e.g. Vero cells and their derivatives, or cell lines from insects, such as e.g. SF-9 cells and their derivatives.

As it is used herein “derivative” and “derivatives” is to be understood as all descendant cell lines that have been derived from them or have emerged from them with modification or further development.

In one preferred embodiment, the secreted recombinant protein of interest is expressed transiently, constitutively and/or inducibly/conditionally.

“Expressed transiently” as used herein is to be understood as an expression that is limited to a certain period of time and a defined duration. For example, transient expression can be generated by means of a vector containing a coding nucleic acid or an RNA, such as an mRNA coding for a nucleic acid, which is introduced into and maintained in the cell for a specific period of time.

“Expressed constitutively” on the other hand, means that the expression of a protein is constant, unchanging or continuous. This can be obtained, for example, by inserting a nucleic acid that codes for the protein to be expressed into the genomic DNA of a cell.

“Expressed inducibly/conditionally” as it is used herein is to be understood as that the expression is conditional and/or activation-dependent. For example, such inducible expression can be triggered by a substance that is added to the cell medium. It is evident to a person skilled in the art that inducible expression can be combined with transient or constitutive expression as defined herein.

In another preferred embodiment, the cell comprises an artificial recombinant polynucleotide sequence encoding the secreted and/or co-expressed recombinant protein of interest, preferably wherein the cell comprises a separate recombinant polynucleotide sequence, an expression cassette or a vector comprising the recombinant polynucleotide sequence encoding the secreted and/or co-expressed recombinant protein of interest.

According thereto, in a further preferred embodiment, the recombinant polynucleotide sequence encoding the secreted recombinant protein of interest comprises integrated optimized CU-rich synonymous codons within the coding sequence. Non-limiting, preferred examples of optimized CU-rich synonymous codons are e.g. Leu: CUG-->CUU, CUC-->CUU; Pro: CCG-->CCU, CCA-->CCU, CCC-->CCU, Ser: AGU-->UCU, UCG-->UCU, UCA-->UCU.

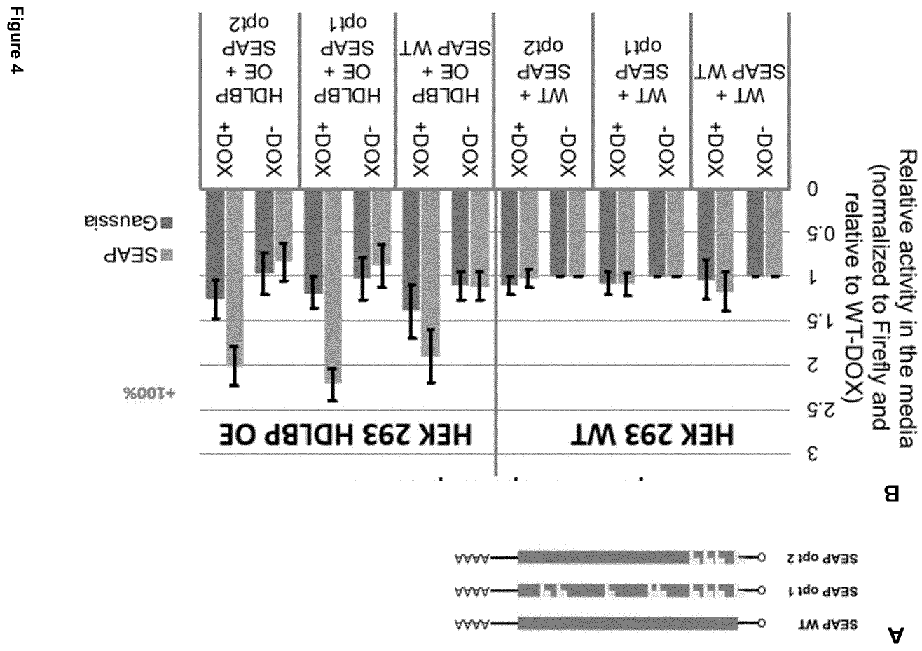

As could be demonstrated, codon-optimization of a gene of interest within the context of the present invention leads to an even more pronounced increase in expression of a secreted protein of interest upon co-expression of HDLBP (cf. FIGS. 4 and 6).

In case of the recombinant polynucleotide sequence being a DNA sequence, the sequence comprises integrated codons which are transcribed to optimized CU-rich synonymous codons within the transcribed mRNA sequence.

CU-rich sequences are known to affect binding abilities of different proteins in the art. In one preferred embodiment, integration of CU-rich synonymous codons within the coding sequence of the protein of interest increases mRNA interactions with HDLBP and may additionally serve to increase its translation and production according to the present invention.

As described above, the cell must take up the recombinant polynucleotide sequence coding for the protein of interest to be produced. According thereto, in a more preferred embodiment, the recombinant polynucleotide sequence is an mRNA sequence.

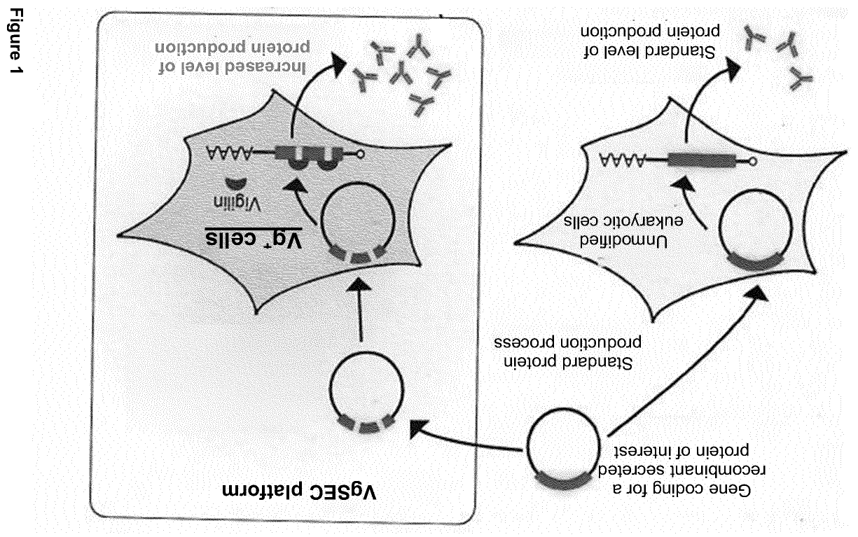

Related thereto, in another preferred embodiment, the method may serve as a platform for improved protein expression (see also FIG. 1). In this regard, two strategies are envisaged herein: First, an established cell line for expression of a specific secreted protein of interest may be modified by introducing a polynucleotide sequence which causes overexpression or forced expression of HDLBP within said cell, thus leading to increased expression of the protein of interest in comparison to the established or parental cell line.

Second, a standard eukaryotic cell line modified to artificially co-and overexpress HDLBP may be used for the introduction of a polynucleotide sequence which encodes a secreted recombinant protein of interest. Said cell line will provide increased levels of expressed secreted protein in comparison to other cell lines with standard or unmodified expression of HDLBP.

Both strategies allow a high degree of flexibility and adaptation to the desired secreted protein of interest.

In another preferred embodiment, the method is carried out in vitro. “Carried out in vitro” as it is used herein, is to be understood as that the method takes place outside a living human or animal organism. Preferably, the method is carried out in a cell culture system.

In one embodiment of the first aspect of the present invention, cells are treated with phytohemagglutinin, testosterone C, beta-estradiol, spermidine, or cholesterol, preferably with phytohemagglutinin or spermidine. Such treatment is able to further increase expression and secretion of a protein of interest by up to 250% in comparison to an untreated control (cf. FIG. 7).

In one particular embodiment, the cells are treated with phytohemagglutinin, preferably with 1 μg/ml to 1 mg/ml phytohemagglutinin, more preferably with 5 μg/ml to 500 μg/ml, even more preferably with 8 μg/ml to 200 μg/ml.

In another particular embodiment, the cells are treated with 0.2 to 10 mM spermidine, preferably with 0.5 to 5 mM, more preferably with 0.8 to 2 mM spermidine.

With an additional treatment of cells with these substances, a further increase in protein secretion by 96% (using 1 mM spermidine) or 95% and up to 250% (using 10 μg/ml or 100 μg/ml phytohemagglutinin, respectively) can be obtained (cf. FIG. 7). Such increase is considered to affect expression and secretion of a protein of interest also in HDLBP-overexpressing cell systems.

In a second aspect of the present invention, provided herein is a eukaryotic cell, wherein the cell is modified to produce a secreted recombinant protein of interest, wherein the cell artificially co-expresses a protein different from the secreted recombinant protein of interest, wherein the amino acid sequence of the co-expressed protein has at least 90% homology to an amino acid sequence according to SEQ ID NO: 1, or wherein the amino acid sequence of the co-expressed protein has at least 90% sequence identity to an amino acid sequence according to SEQ ID NO: 2.

In one embodiment, the eukaryotic cell comprises a recombinant polynucleotide sequence encoding the secreted recombinant protein of interest, preferably the cell comprises a non-genomic recombinant polynucleotide sequence, an expression cassette or a vector comprising the recombinant polynucleotide sequence encoding the secreted recombinant protein of interest.

In a specific embodiment, the recombinant polynucleotide sequence in the eukaryotic cell encoding the secreted recombinant protein of interest comprises integrated CU-rich synonymous codons within the coding sequence.

In an embodiment of the present invention, the recombinant polynucleotide sequence is an mRNA sequence.

The eukaryotic cell according to the present invention may be selected from the group consisting of: CHO cells, BHK 21 cells, HEK293 cells, C127, Sp2/0, YB2/0, SF-9 cells, NS0 cells, Vero cells, and any derivatives thereof.

In one embodiment of the eukaryotic cell of the present invention, the secreted recombinant protein of interest is suitable for protein-based therapies, preferably the secreted recombinant protein is selected from the group consisting of: an antibody, such as a therapeutic antibody, or an antigen-binding fragment thereof, or a nanobody, or an antibody-drug conjugate, or a recombinant fusion protein, or an exosome, or a cytokine, such as IFN-B, or a hormone, such as insulin, or a hormone analogue, such as a GnRH analogue.

In a third aspect of the present invention, the use of a eukaryotic cell according to the second aspect of the present invention is provided in a method of producing a secreted recombinant protein of interest in vitro.

All embodiments of the present invention as disclosed and described herein are deemed to be combinable in any combination, unless the skilled person considers such a combination to not make any technical sense.

The invention is now further explained by individual examples which are intended to illustrate but not to limit the present invention.

### EXAMPLES

Based on previous findings, the function of HDLBP in ER-associated translation and secretion was examined by the inventors. Thus, the process of active translation in the absence of HDLBP was first studied by generating two CRISPR/Cas9 HDLBP knockout (KO) cell-lines.

HEK293 and A549 HDLBP knockout cell lines were produced using the Edit-R CRISPR-Cas9 Gene Engineering kit (Dharmacon) according to manufacturer's instructions. Briefly, transfections of synthetic tracrRNA (U-002000-05), hCMV-PuroR-Cas9 (U-005100-120) and pre-designed HDLBP crRNA (either guide 1 (CR-019956-01-0005) or guide 2 (CR-019956-04-0005)) or a non-targeting control (U-007501-05) were carried out using DharmaFECT Duo transfection reagent (Dharmacon, T2010-01) in a 12-well plate.

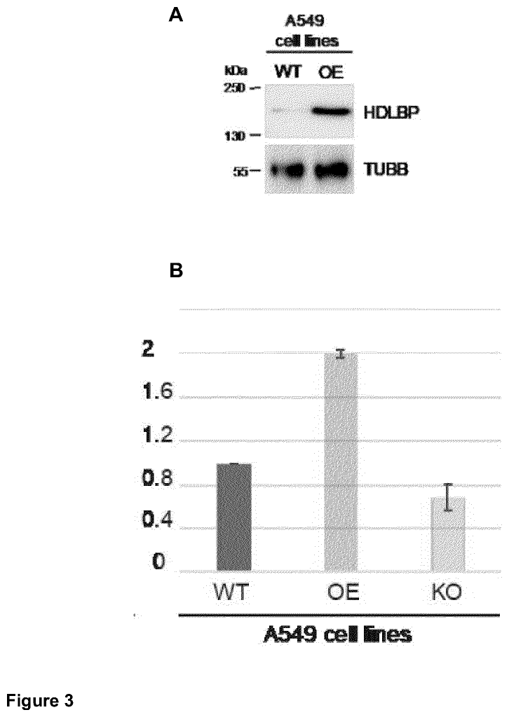

After 2 days, cells were reseeded to a 10 cm dish and treated with puromycin (2 μg/ml for HEK293 cells and 1 μg/ml for A549 cells). The surviving colonies were picked and Western analysis was performed (see, for example, FIG. 3A for an A 549 KO cell line).

HEK293 Flp-In T-REX (HEK293) (Thermo Fisher Scientific), HEK293 stable cell lines and A549 cells were cultured in standard Dulbecco's modified Eagle's medium (DMEM, Thermo Fisher Scientific) supplemented with 10% fetal bovine serum (FBS, Sigma-Aldrich) and 1% L-glutamine (200 mM, Thermo Fisher Scientific).

The HEK293 HDLBP knockout cells showed no apparent growth defects and electron microscopy imaging of the ER revealed no morphology changes (data not shown). Absence of HDLBP generally resulted in a decrease in protein synthesis of proteins encoded by membrane-bound mRNAs and the extent of decrease depended on the level of HDLBP crosslinking to such an mRNA (data not shown).

Therefore, it could be established that HDLBP is required for efficient protein synthesis of its target mRNAs.

In order to understand if HDLBP is influencing the secretion of certain proteins, secreted Gaussia luciferase (Gluc) and alkaline phosphatase (SEAP) were used as reporter proteins and expressed in HEK293 cells in WT (wild-type) and OE (overexpression) conditions to quantify enzyme activity in the culture medium.

Induction of the stable cell lines was achieved by adding 1 μg/ml of doxycycline to the culture medium and incubation for 16 h.

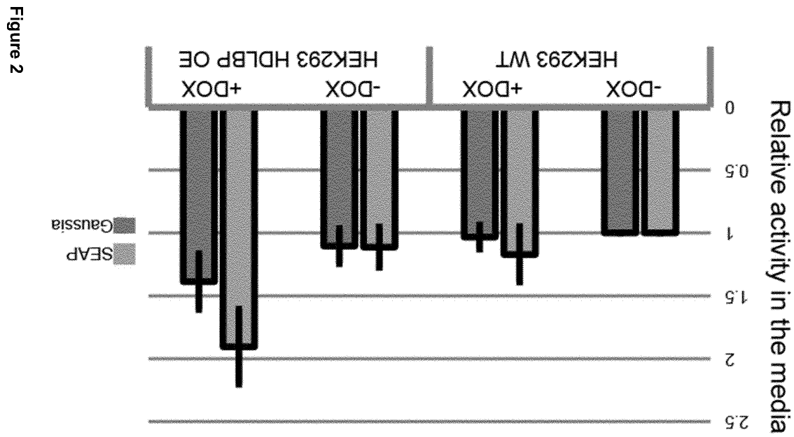

As expected, comparison of the parental WT cells, either uninduced or induced with doxycycline, showed minimal changes in secretion of SEAP and Gaussia luciferase reporter proteins (see FIG. 2).

In contrast, inducible overexpression of HDLBP by doxycycline induction in piggybac transfected HEK293 cells increased SEAP and Gaussia luciferase in the growth medium by about 1.8-fold and 1.4-fold, respectively (FIG. 2).

In HDLBP knockout cell lines, Gluc and SEAP activity was significantly decreased in comparison to HDLBP WT by 20-40%, showing that HDLBP depletion reduces secretion of the two reporter proteins (exemplarily shown for SEAP measurements in A549 cells in FIG. 3B).

Since the depletion of HDLBP reduced secretion, the impact of HDLBP overexpression was also tested. To this end, HDLBP was stably overexpressed in A549 cells using a piggybac transposon carrying a doxycycline-inducible HDLBP.

By direct comparison, it could thus be demonstrated that overexpression of HDLBP in A549 cells leads to increased SEAP secretion of about 2-fold, whereas knockout of HDLBP in A549 cell reduced secretion by about 30%, confirming that HDLBP expression levels directly influences the extent of protein secretion (see FIG. 3B).

Based on these findings, the function of HDLBP in secretion of a therapeutic protein, EPO-Fc, was examined by the inventors. CHO cells were stably transfected with an inducible HLDBP construct by piggyback transposition. These HDLBP overexpressing CHO cells were further stably transfected with a construct allowing inducible EPO-Fc or codon-optimized inducible EPO-Fc opt expression. Parental CHO cells were stably transfected with a construct allowing inducible EPO-Fc or codon-optimized inducible EPO-Fc opt expression to examine the effect of HDLBP expression on the secretion of EPO-Fc and EPO-Fc opt (FIG. 5).

Intracellular expression of HDLBP and EPO-Fc or EPO-Fc opt in respective CHO cell lines was examined by Western analysis after 24 hours of induction with doxycycline with anti-HDLBP and anti-Fc antibodies. Equal loading of protein samples was controlled for by Western analysis with an anti-GAPDH antibody and Ponceau staining (FIG. 6A).

To determine the effect of HDLBP overexpression (HDLBP OE) on the production/secretion of EPO-Fc in the cell medium compared to control CHO cells (control) only expressing EPO-Fc or EPO-Fc opt genes, the amount of EPO-Fc in the cell medium was measured by ELISA assay with an anti-Fc antibody. Relative amount of secreted EPO-Fc was determined by normalizing EPO-Fc protein amount to control cells (FIG. 6B). Overexpression of HDLBP increased EPO-Fc secretion by 75%. Interestingly, codon optimization resulted in a 115% increase without overexpression of HDLBP and 290% with HDLBP overexpression compared to parental control cell expressing the non-optimized EPO-Fc construct.

To examine the effect of natural compounds on EPO-Fc expression, CHO cells stably transfected with EPO-Fc expression construct, were treated with phytohemagglutinin, testosterone C, beta-estradiol, spermidine, or cholesterol with concentrations indicated for 24 hours. Expression of EPO-Fc was induced by addition of doxycycline with the addition of natural compounds.

Intracellular expression of HDLBP and EPO-Fc in respective CHO cell lines was examined by Western analysis after 24 hours of compound treatment and of induction with doxycycline with anti-HDLBP and anti-Fc antibodies. Equal loading of protein samples was controlled for by Western analysis with an anti-GAPDH antibody and Ponceau staining (FIG. 7A).

To determine the effect of natural compounds on the production/secretion of EPO-Fc in the cell medium compared to untreated EPO-Fc expressing CHO cells (control), the amount of EPO-Fc in the cell medium was measured by ELISA assay with an anti-Fc antibody. Relative amount of secreted EPO-Fc from treated cells was determined by normalizing EPO-Fc protein amount to that of untreated control cells (FIG. 7B). Interestingly, treatment with phytohemagglutinin and spermidine increased EPO-Fc secretion by 250% and 96%, respectively.

Taken together, these results demonstrate that HDLBP directly influences the extent of secretion of secretory proteins in eukaryotic expression systems. Even more, it could be shown that overexpression/forced expression of HDLBP in a eukaryotic cell leads to a significant increase of production of recombinant secretory proteins.

1. A method for producing a secreted recombinant protein of interest in a cell, wherein the method comprises artificially co-expressing a protein different from the secreted recombinant protein of interest, wherein the amino acid sequence of the co-expressed protein has at least 90% sequence identity to an amino acid sequence according to SEQ ID NO: 1, or wherein the amino acid sequence of the co-expressed protein has at least 90% sequence identity to an amino acid sequence according to SEQ ID NO: 2.

2. The method according to claim 1, wherein the amino acid sequence of the co-expressed protein has at least 95% sequence identity to an amino acid sequence according to SEQ ID NO: 1 or according to SEQ ID NO: 2.

3. The method according to claim 1, wherein the co-expressed protein comprises an amino acid sequence according to SEQ ID NO: 1 or according to SEQ ID NO: 2.

4. The method according to claim 1, wherein the secreted recombinant protein of interest is suitable for protein-based therapies, preferably wherein the secreted recombinant protein is selected from the group consisting of: an antibody, such as a therapeutic antibody, or an antigen-binding fragment thereof, or a nanobody, or an antibody-drug conjugate, or a recombinant fusion protein, or an exosome, or a cytokine, such as IFN-|3, or a hormone, such as insulin, or a hormone analogue, such as a GnRH analogue, more preferably wherein the secreted recombinant protein of interest is EPO-Fc.

5. The method according to claim 1, wherein the polynucleotide encoding the secreted recombinant protein of interest has at least one codon coding for Pro, Ser or Leu which has been optimized to have an increased CU content in comparison to the wild-type sequence, preferably wherein at least one codon coding for Leu has been optimized to CUU, or at least one codon coding for Pro has been optimized to CCU, or at least one codon coding for Ser has been optimized to UCU, more preferably wherein at least one CUG codon coding for Leu has been optimized to CUU, or at least one CCC codon coding for Pro has been optimized to CCU, or at least one UCA codon coding for Ser has been optimized to UCU, even more preferably at least two or at least three or at least four codons have been optimized in this fashion.

6. The method according to claim 1, wherein the cell is a eukaryotic cell.

7. The method according to claim 1, wherein the cell is selected from the group consisting of: CHO cells, BHK 21 cells, HEK293 cells, C127, A549, Sp2/0, YB2/0, SF-9 cells, NS0 cells, Vero cells, PER. C6, and any derivatives thereof.

8. The method according to claim 1, wherein the method is carried out in vitro.

9. The method according to claim 1, wherein the cells are additionally treated with phytohemagglutinin, testosterone C, beta-estradiol, spermidine, or cholesterol, preferably wherein the cells are additionally treated with phytohemagglutinin or spermidine.

10. A eukaryotic cell, wherein the cell is modified to produce a secreted recombinant protein of interest, wherein the cell artificially co-expresses a protein different from the secreted recombinant protein of interest, wherein the amino acid sequence of the co-expressed protein has at least 90% homology to an amino acid sequence according to SEQ ID NO: 1, or wherein the amino acid sequence of the co-expressed protein has at least 90% sequence identity to an amino acid sequence according to SEQ ID NO: 2.

11. The eukaryotic cell of claim 10, wherein the secreted recombinant protein of interest is expressed transiently, constitutively and/or inducibly/conditionally.

12. The eukaryotic cell of claim 10, wherein the cell comprises a recombinant polynucleotide sequence encoding the secreted recombinant protein of interest, preferably wherein the cell comprises a non-genomic recombinant polynucleotide sequence, an expression cassette or a vector comprising the recombinant polynucleotide sequence encoding the secreted recombinant protein of interest.

13. The eukaryotic cell of claim 12, wherein the recombinant polynucleotide sequence encoding the secreted recombinant protein of interest comprises integrated CU-rich synonymous codons within the coding sequence.

14. The eukaryotic cell of claim 12, wherein the recombinant polynucleotide sequence is an mRNA sequence.

15. The eukaryotic cell of claim 10, wherein the cell is selected from the group consisting of: CHO cells, BHK 21 cells, HEK293 cells, C127, Sp2/0, YB2/0, SF-9 cells, NS0 cells, Vero cells, and any derivatives thereof.

16. The eukaryotic cell of claim 10, wherein the secreted recombinant protein of interest is suitable for protein-based therapies, preferably wherein the secreted recombinant protein is selected from the group consisting of: an antibody, such as a therapeutic antibody, or an antigen-binding fragment thereof, or a nanobody, or an antibody-drug conjugate, or a recombinant fusion protein, or an exosome, or a cytokine, such as IFN-|3, or a hormone, such as insulin, or a hormone analogue, such as a GnRH analogue.

17. Use of the eukaryotic cell according to claim 10 in a method of producing a secreted recombinant protein of interest in vitro.

18. The method according to claim 1, wherein the amino acid sequence of the co-expressed protein has at least 98% sequence identity to an amino acid sequence according to SEQ ID NO: 1 or according to SEQ ID NO: 2.

19. The method according to claim 1, wherein the amino acid sequence of the co-expressed protein has at least 99% sequence identity to an amino acid sequence according to SEQ ID NO: 1 or according to SEQ ID NO: 2.
